SEQUENCE LISTING (103 sequences)

SEQ ID NO: 1 - protein, 13 aa, Artificial Sequence
  Description of Artificial Sequence LLP Boxmotif
XRXXXXGXXXXHX

SEQ ID NO: 2 - protein, 13 aa, Artificial Sequence
  Description of Artificial Sequence consensus sequence LLP boxmotif
KRXXXXGXXXXHX

SEQ ID NO: 3 - protein, 13 aa, Brassica napus
  /note=”LLP boxmotif“
KRIIPTGPNPLHN

SEQ ID NO: 4 - protein, 13 aa, Arabidopsis thaliana
  /note=”LLP boxmotif“
KRLVPSGPNPLHN

SEQ ID NO: 5 - protein, 13 aa, Arabidopsis thaliana
  /note=”LLP boxmotif“
KRLVPSGPNPLHH

SEQ ID NO: 6 - protein, 13 aa, Arabidopsis thaliana
  /note=”LLP boxmotif“
KRRVPSGPNPLHN

SEQ ID NO: 7 - protein, 13 aa, Arabidopsis thaliana
  /note=”LLP boxmotif“
KRRVPSGPNPLHH

SEQ ID NO: 8 - protein, 13 aa, Arabidopsis thaliana
  /note=”LLP boxmotif“
KRLVHSGPNPLHN

SEQ ID NO: 9 - protein, 13 aa, Arabidopsis thaliana
  /note=LLP boxmotif”
KRVIPSGPNPLHN

SEQ ID NO: 10 - protein, 13 aa, Arabidopsis thaliana
  note=“LLP boxmotif”
KRKVPSGPNPLHN

SEQ ID NO: 11 - protein, 13 aa, Arabidopsis thaliana
  /note=“LLP boxmotif”
KRSIPSGPNPLHN

SEQ ID NO: 12 - protein, 13 aa, Arabidopsis thaliana
  /note=“LLP boxmotif”
KRKVPNGPNPLHN

SEQ ID NO: 13 - protein, 13 aa, Arabidopsis thaliana
  /note=“LLP boxmotif”
KRKVPRGPNPLHN

SEQ ID NO: 14 - protein, 13 aa, Arabidopsis thaliana
  /note=“LLP boxmotif”
KRSIPTGPNPLHN

SEQ ID NO: 15 - protein, 13 aa, Arabidopsis thaliana
  /note=“LLP boxmotif”
ERLVPSGPNPLHN

SEQ ID NO: 16 - protein, 13 aa, Arabidopsis thaliana
  /note=“LLP boxmotif”
ERLVPSGPNPLHH

SEQ ID NO: 17 - protein, 13 aa, Arabidopsis thaliana
  /note=“LLP boxmotif”
ARLVPSGPNPLHN

SEQ ID NO: 18 - protein, 13 aa, Arabidopsis thaliana
  /note=“LLP boxmotif”
ARLVPKGPNPLHN

SEQ ID NO: 19 - protein, 13 aa, Arabidopsis thaliana
  /note=“LLP boxmotif”
KRVVPSGPNPLHN

SEQ ID NO: 20 - protein, 13 aa, Arabidopsis thaliana
  /note=“LLP boxmotif”
KRVVHTGPNPLHN

SEQ ID NO: 21 - protein, 13 aa, Arabidopsis thaliana
  /note=“LLP boxmotif”
KRRVPSGPNPLHN

SEQ ID NO: 22 - protein, 13 aa, Arabidopsis thaliana
  /note=“LLP boxmotif”
KRRVFSGPNPLHN

SEQ ID NO: 23 - protein, 13 aa, Arabidopsis thaliana
  /note=“LLP boxmotif”
KRKVPKGPNPLHN

SEQ ID NO: 24 - protein, 13 aa, Arabidopsis thaliana
  /note=“LLP boxmotif”
KRKVKSGPNSLHN

SEQ ID NO: 25 - protein, 13 aa, Arabidopsis thaliana
  /note=“LLP boxmotif”
KRLSPGGPNPLHN

SEQ ID NO: 26 - protein, 13 aa, Arabidopsis thaliana
  /note=“LLP boxmotif”
MRLVPSGPNPLHN

SEQ ID NO: 27 - protein, 4 aa, Artificial Sequence
  Description of Artificial Sequence submotif
NPLH

SEQ ID NO: 28 - protein, 4 aa, Artificial Sequence
  Description of Artificial Sequence submotif
DPLH

SEQ ID NO: 29 - protein, 4 aa, Artificial Sequence
  Description of Artificial Sequence submotif
NPRH

SEQ ID NO: 30 - protein, 4 aa, Artificial Sequence
  Description of Artificial Sequence submotif
DPRH

SEQ ID NO: 31 - protein, 12 aa, Artificial Sequence
  Description of Artificial Sequence LLP box conserved motif
KRXXPXGPXPLH

SEQ ID NO: 32 - DNA, 14 bp, Artificial Sequence
  Description of Artificial Sequence primer
ttttttttttttmn

SEQ ID NO: 33 - DNA, 29 bp, Artificial Sequence
  Description of Artificial Sequence adapter primer AP1
ccattcttcatcagcaaactccgaaatga

SEQ ID NO: 34 - DNA, 28 bp, Artificial Sequence
  Description of Artificial Sequence adapter primer AP2
cagaaaagaggaagccaatatcaaactc

SEQ ID NO: 35 - DNA, 31 bp, Artificial Sequence
  Description of Artificial Sequence P312-1 FEATURE
cgctctagagttctatctttgtcaaaaaaaa

SEQ ID NO: 36 - DNA, 26 bp, Artificial Sequence
  Description of Artificial Sequence P312-2 FEATURE
atataagcttactatagggcacgcgt

SEQ ID NO: 37 - DNA, 5 bp, Artificial Sequence
  Description of Artificial Sequence CA AT-box FEATURE
ccaat

SEQ ID NO: 38 - DNA, 531 bp, Brassica napus
  Description of Combined DNA/RNA Molecule Br assica napus
taacatgcttcctttattttagtgcacccgtagcttgtatataaaattttatccacacaaattccttttgctccttttatcaaacgtcgctatttgtatcagtctacctatctgctttcatattgtatctcccgcagccaaacaaaaactttttttttgacaaagatagaacatgaagatcaagagtttgatattggcttcctcttttctgattcttgccttcattcatcactcagaatcagcttcatttcggagtttgctgatgaagaatggattgtacgaagaagaagaagcaaaaattctattgggcgactccaaagaaacgataactaattctacagctttggagtctaaacggataattccgacgggtccaaatccacttcacaacaggtaactttgatcatttaagaacaagatatgttgtgagtatgtctcttcctctgtttttgttacaagtatctatcttactgtgtaatgtaatggtgaatgtataatacattttctaattaaattaccttatttaaaaaa

SEQ ID NO: 39 - protein, 99 aa, Brassica napus
  Description of Combined DNA/RNA Molecule Brassica napus
LVYKILSTQIPFAPFIKRRYLYQSTYLLSYCISRSQTKTFFLTKIEHEDQEFDIGFLFSDSCLHSSLRISFISEFADEEWIVRRRRSKNSIGRLQRNDN

SEQ ID NO: 40 - DNA, 374 bp, Brassica napus
  Description of Combined DNA/RNA Molecule Brassica napus
caaagatagaacatgaagatcaagagtttgatgttggcttcctcttttctgattcttgccttcattcatcactcagaatcagcttcatttcggagtttgctgatgaagaatgtattgtacgaacaagaagaagcaaaaattctattgggcgactccaaagaaacgataactaattctacagctttggagtctaaacggataattccgacgggtccaaatccacttcacaacaggtaaatttgatcatttaagaacaagatatgttgtgagtatgtctcttcctcctctgtttttgttacaagtatctatcttactgtgtaatgtaatggtgaatgtataagacattttctaattaaattaccttatttaaaaaa

SEQ ID NO: 41 - protein, 75 aa, Brassica napus
  Description of Combined DNA/RNA Molecule Brassica napus
NMKIKSLMLASSFLILAFIHHSESASFRSLLMKNVLYEQEEAKILLGDSKETITNSTALESKRIIPTGPNPLHNR

SEQ ID NO: 42 - protein, 99 aa, Arabidopsis thaliana
  /note=“LLP box genes”
MATSNDQTNTKSSHSRTLLLLFIFLSLLLFSSLTIPMTRHQSTSMVAPFKRVLLESSVPASSTMDLRPKASTRRSRTSRRREFGNDAHEVPSGPNPISN

SEQ ID NO: 43 - protein, 107 aa, Arabidopsis thaliana
  /note=“LLP box genes”
MKTNRNRPINILIVFFLLTTARAATRNWTNRTHRTVPKVQHAYYAYPHRSCESFSRPYARSMCIELERIHRSSRQPLFSPPPPPTEIDQRYGVEKRLVPSGPNPLHN

SEQ ID NO: 44 - protein, 120 aa, Arabidopsis thaliana
  /note=“LLP box genes”
MTMTHLNRLILISLLFVSLLLKSSTASSTVVDEGNRTSRNFRYRTHRFVPRFNHHPYHVTPHRSCDSFIRPYARSMCIELQRIHRSSRKQPLLSPPPPEIDPRYGVDKRLVPSGPNPLHN

SEQ ID NO: 45 - protein, 74 aa, Arabidopsis thaliana
  /note=“LLP box genes”
MKIKGLMILASSLLILAFIHQSESASMRSLLMNNGSYEEEEQVLKYDSMGTIANSSALDSKRVIPTGPNPLHNR

SEQ ID NO: 46 - protein, 74 aa, Arabidopsis thaliana
  /note=“LLP box genes”
MKIKSLILASSFLILAFIHHSESASFRSLLMKNGLYEEEEAKILLGDSKETITNSTALESKRIIPTGPNPLHNR

SEQ ID NO: 47 - protein, 80 aa, Arabidopsis thaliana
  /note=“LLP box genes”
MASFKLWVCLILLLLEFSVHQCRPLVAEESPSDSGNIRKIMRELLKRSEELKVRSKDGQTVLGTLDSKRLSPGGPDPRHH

SEQ ID NO: 48 - protein, 83 aa, Arabidopsis thaliana
  /note=“LLP box genes”
MASLKLWVCLVLLLVLELTSVHECRPLVAEERFSGSSRLKKIRRELFERLKEMKGRSEGEETILGNTLDSKRLSPGGPDPRHH

SEQ ID NO: 49 - protein, 97 aa, Arabidopsis thaliana
  /note=“ LLP box genes”
MKRDVLIIVIFTVLVLIIISRSSSIQAGRFMTTGRNRNLSVARSLYYKNHHKVVITEMSNFNKVRRRSSRFRRKTDGDEEEEEKRSIPTGPNPLHNK

SEQ ID NO: 50 - protein, 80 aa, Arabidopsis thaliana
  /note=“LLP box genes”
MKVWSQRLSFLIVMIFILAGLHSSSAGRKLPSMTTTEEFQRLSFDGKRILSEVTADKKYDRIYGASARLVPKGPNPLHNK

SEQ ID NO: 51 - protein, 107 aa, Arabidopsis thaliana
  /note=“LLP box genes”
MATTRVSHVLGFLLWISLLIFVSIGLFGNFSSKPINPFPSPVITLPALYYRPGRRALAVKTFDFTPFLKDLRRSNHRKALPAGGSEIDPRYGVEKRLVPSGPNPLHH

SEQ ID NO: 52 - protein, 109 aa, Arabidopsis thaliana
  /note=“LLP box genes”
MALKFSQILFIVLWLSLFFLLLHHLYSLNFRRLYSLNAVEPSLLKQHYRSYRLVSRKVLSDRFDFTPFHSRDNSRHNHRSGEQYDGDEIDPRYGVEKRRVPSGPNPLHH

SEQ ID NO: 53 - protein, 99 aa, Arabidopsis thaliana
  /note=“LLP box genes”
MTKQPKPCSFLFHISLLSALFVFLLISFAFTTSYKLKSGINSLGHKRILASNFDFTPFLKNKDRTQRQRQSPSLTVKENGFWYNDEERVVPSGPNPLHH

SEQ ID NO: 54 - protein, 103 aa, Arabidopsis thaliana
  /note=“LLP box genes”
MGNYYSRRKSRKHITTVALIILLLLLFLFLYAKASSSSPNIHHHSTHGSLKKSGNLDPKLHDLDSNAASSRGSKYTNYEGGGEDVFEDGKRRVFTGPNPLHNR

SEQ ID NO: 55 - protein, 79 aa, Arabidopsis thaliana
  /note=“ LLP box genes”
MTMCFFLFFFVFYVSFQIVLSSSASVGYSRLHLVASPPPPPPRKALRYSTAPFRGPLSRDDIYGDDKRVVHTGPNPLHN

SEQ ID NO: 56 - protein, 103 aa, Arabidopsis thaliana
  /note=“LLP box genes”
MEACSRKRRRRRAYTTSTTGYAAVFFCGIFVFAQFGISSSALFAPDHYPSLPRKAGHFHEMASFQAPKATVSFTGQRREEENRDEVYKDDKRLVHTGPNPLHN

SEQ ID NO: 57 - protein, 83 aa, Arabidopsis thaliana
  /note=“LLP box genes”
MKNKNMNPSRPRLLCLIVFLFLVIVLSKASRIHVERRRFSSKPSGENREFLPSQPTFPVVDAGEILPDKRKVKTGSNPLHNKR

SEQ ID NO: 58 - protein, 86 aa, Arabidopsis thaliana
  /note=“LLP box genes”
MGGNGIRALVGVIASLGLIVFLLVGILANSAPSVPSSENVKTLRFSGKDVNLFHVSKRKVPNGPDPIHNRFLSLLSRIFNLLLLLL

SEQ ID NO: 59 - protein, 118 aa, Arabidopsis thaliana
  /note=“LLP box genes”
MRNNHSLRLQLWFRTLFTVGVVTLLMIDAFVLQNNKEDDKTKEITTAVNMNNSDAKEIQQELEDGSRNDDLSYVASKRKVPRGPDPIHNRFLLLSRFILSLLTNPYPYLHICVLDVSV

SEQ ID NO: 60 - protein, 86 aa, Arabidopsis thaliana
  /note=“LLP box genes”
MKVLKRDSMLLLITLYFLLTTSMARQDPFLVGVEKDVVPAGTDLKQNKAKPHLPNLFRTMRRVPTGPNPLHHISPPQPGSLNYARN

SEQ ID NO: 61 - protein, 96 aa, Arabidopsis thaliana
  /note=“LLP box genes”
MDSKSFVLLLLLFCFLFLHDASDLTQAHAHVQGLSNRKMMMMKMESEWVGANGEAEKAKTKGLGLHEELRTVPSGPDPLHHHVNPPRQPRNNFQLP

SEQ ID NO: 62 - protein, 75 aa, Arabidopsis thaliana
  /note=“LLP box genes”
MVILLSYMLHLFCVYSRVGAIRIFPETPASGKRQEEDLMKKYFGAGKFPPVDSFVGKGISESKRIVPSCPDPLHN

SEQ ID NO: 63 - protein, 62 aa, Arabidopsis thaliana
  /note=“LLP genes identified in higher plants”
MASFKLWVCLILLLLEFSVHQCRPLVAEESPSDKVRSKDGQTVLGTLDSKRLSPGGPDPRHH

SEQ ID NO: 64 - protein, 56 aa, Brassica napus
  /note=“LLP genes identified in higher plants”
MKIKSLILASSFLILAFIHHSESASFLGDSKETITNSTALESKRIIPTGPNPLHNR

SEQ ID NO: 65 - protein, 57 aa, Arabidopsis thaliana
  /note=“LLP genes identified in higher plants”
MKIKGLMILASSLLILAFIHQSESASMKYDSMGTIANSSALDSKRVIPTGPNPLHNR

SEQ ID NO: 66 - protein, 75 aa, Arabidopsis thaliana
  /note=“LLP genes identified in higher plants”
MATSNDQTNTKSSHSRTLLLLFIFLSLLLFSSLTIPMTRHQSRPKASTRRSRTSRRREFGNDAHEVPSGPNPISN

SEQ ID NO: 67 - protein, 84 aa, Arabidopsis thaliana
  /note=“LLP genes identified in higher plants”
MKTNRNRPINILIVFFLLTTARAATRNWTNRTHRTVPKVQHAYYAYPHRSCQPLFSPPPPPTEIDQRYGVEKRLVPSGPNPLHN

SEQ ID NO: 68 - protein, 97 aa, Arabidopsis thaliana
  /note=“LLP genes identified in higher plants”
MTMTHLNRLILISLLFVSLLLKSSTASSTVVDEGNRTSRNFRYRTHRFVPRFNHHPYHVTPHRSCKQPLLSPPPPEIDPRYGVDKRLVPSGPNPLHN

SEQ ID NO: 69 - protein, 116 aa, Zea mays
  /note=“LLP genes identified in higher plants”
MASRMGMVAIVSLFVCALAASTSVNANVWQTDDIPVVNSNMVRHSNMERQQQQGGFIGGDRKRTVPSGPDHMHHSIPSHTPQHPPVYVQALYEDDRSRTSSGPSKSIGPPPLSDRY

SEQ ID NO: 70 - protein, 110 aa, Zea mays
  /note=“LLP genes identified in higher plants”
MASRMGMVAIMSLFVYAIVVPTSVNANAWQTDDKPGVNRNMEMQQQQGGFIGGDRKRTVPSGPNHKHNNIPSHTPHHPPSYVQALYEDDRTITSPGPSKSIGPPPLPDRY

SEQ ID NO: 71 - protein, 82 aa, Zea mays
  /note=“LLP genes identified in higher plants”
TRTDDKPGVNRNMEMQQQQGGFIGDREKRPVPSGPDPIHHSIPSHAPQHPPSYGKAPYEDDKSIASPGLSNLIGPPPFLDRY

SEQ ID NO: 72 - protein, 73 aa, Arabidopsis thaliana
  note=“LLP genes identified in higher plants”
MKRDVLIIVIFTVLVLIIISRSSSIQAGRFMTTGRNRNLSRRRSSRFRRKTDGDEEEEEKRSIPTGPNPLHNK

SEQ ID NO: 73 - protein, 55 aa, Soybean glycine max
  /note=“LLP genes identified in higher plants”
MKHFHLFLFLALLFLTPRVHAIRWANSPIRSSREREFMSEKRRVPTGSNPLHNKR

SEQ ID NO: 74 - protein, 42 aa, Lycopersicon esculentum
  /note=“LLP genes identified in higher plants”
WIPRAAGSPRKESVIFQPLPTSFKFENRRVPTGSNPLHNKKR

SEQ ID NO: 75 - protein, 63 aa, Arabidopsis thaliana
  /note=“LLP genes identified in higher plants”
MKNKNMNPSRPRLLCLIVFLFLVIVLSKASLPSQPTFPVVDAGEILPDKRKVKTGSNPLHNKR

SEQ ID NO: 76 - protein, 57 aa, Arabidopsis thaliana
  /note=“LLP genes identified in higher plants”
MKVWSQRLSFLIVMIFILAGLHSSRILSEVTADKKYDRIYGASARLVPKGPNPLHNK

SEQ ID NO: 77 - protein, 78 aa, Arabidopsis thaliana
  /note=“LLP genes identified in higher plants”
MGNYYSRRKSRKHITTVALIILLLLLFLFLYAKASSSSPNIHHHSRGSKYTNYEGGGEDVFEDGKRRVFTGPNPLHNR

SEQ ID NO: 78 - protein, 59 aa, Arabidopsis thaliana
  /note=“LLP genes identified in higher plants”
MTMCFFLFFFVFYVSFQIVLSSSASVGYSTAPFRGPLSRDDIYGDDKRVVHTGPNPLHN

SEQ ID NO: 79 - protein, 126 aa, Medicago
  /note=“LLP genes identified in higher plants”
FSFLSFHFYTWFNCFLQAQLPPYKRFSLSNSISNHYCNCSIKDFEVSATVLPRYKGFSLHNSIATVIGILKSPQQYCNRNFDILFPHFSAIFTSSASSRKNLSRSSNPELEDKRGVPSGANPLHNR

SEQ ID NO: 80 - protein, 77 aa, Arabidopsis thaliana
  /note=“LLP genes identified in higher plants”
MEACSRKRRRRRAYTTSTTGYAAVFFCGIFVFAQFGISSSALFAPSFTGQRREEENRDEVYKDDKRLVHTGPNPLHN

SEQ ID NO: 81 - protein, 84 aa, Arabidopsis thaliana
  /note=“LLP genes identified in higher plants”
MALKFSQILFIVLWLSLFFLLLHHLYSLNFRRLYSLNAVEPSLLKQHYRSYNHRSGEQYDGDEIDPRYGVEKRRVPSGPNPLHH

SEQ ID NO: 82 - protein, 85 aa, Arabidopsis thaliana
  /note=“LLP genes identified in higher plants”
MATTRVSHVLGFLLWISLLIFVSIGLFGNFSSKPINPFPSPVITLPALYYRSNHRKALPAGGSEIDPRYGVEKRLVPSGPNPLHH

SEQ ID NO: 83 - protein, 72 aa, Arabidopsis thaliana
  /note=“LLP genes identified in higher plants”
MTKQPKPCSFLFHISLLSALFVFLLISFAFTTSYKLKSGRQRQSPSLTVKENGFWYNDEERVVPSGPNPLHH

SEQ ID NO: 84 - protein, 62 aa, Zea mays
  /note=“LLP genes identified in higher plants”
MGLPQQSLVRLMVVVVLTVSFLGAEQRGLFCHGRLGWRRVVQVQDLSEAKRRVPQGSNPLHN

SEQ ID NO: 85 - protein, 69 aa, Oryza sativa
  /note=“LLP genes identified in higher plants”
MLRSRKSRVMVMLVTAALLLTDMAGVSYGRRLIPDLDPSDSGHHVGDEYRSMHAVSKRLVPQGPNPLHN

SEQ ID NO: 86 - protein, 94 aa, Oryza sativa
  /note=“LLP genes identified in higher plants”
MKPSSSSVGVCTLLVAVASLQLLLVVAVAASAKTAPAMPDEEFLGRLCDQQQGATRRRLPWCPMPPPSRAGEEIDARYDVSKRVVPSGPNPLHN

SEQ ID NO: 87 - protein, 70 aa, Arabidopsis thaliana
  /note=“LLP genes identified in higher plants”
MKVLKRDSMLLLITLYFLLTTSMARTDLKQNKAKPHLPNLFRTMRRVPTGPNPLHHISPPQPGSLNYARN

SEQ ID NO: 88 - protein, 89 aa, Oryza sativa
  /note=“LLP genes identified in higher plants”
MRPARRGWGVGALARAFLVLLLLLLAAATTTTIGFGCRGAEAIRVIPPHGPAPGAMPVVGTRPVPALSPAADEESKRRIPSCPDPLHNR

SEQ ID NO: 89 - protein, 72 aa, Oryza sativa
  /note=“LLP genes identified in higher plants”
MPPPPATTPLPRRRLALILCLAWALWLHGGGGGISVPAAAPRWSSSSASEAAARFADDKRRIPSCPDALHNR

SEQ ID NO: 90 - protein, 54 aa, Arabidopsis thaliana
  /note=“LLP genes identified in higher plants”
MVILLSYMLHLFCVYSRVGAIRGAGKFPPVDSFVGKGISESKRIVPSCPDPLHN

SEQ ID NO: 91 - protein, 72 aa, Arabidopsis thaliana
  /note=“LLP genes identified in higher plants”
MDSKSFVLLLLLFCFLFLHDASDLTGANGEAEKAKTKGLGLHEELRTVPSGPDPLHHHVNPPRQPRNNFQLP

SEQ ID NO: 92 - protein, 69 aa, Arabidopsis thaliana
  /note=“LLP genes identified in higher plants”
MGGNGIRALVGVIASLGLIVFLVKTLRFSGKDVNLFHVSKRKVPNGPDPIHNRFLSLLSRIFNLLLLLL

SEQ ID NO: 93 - protein, 90 aa, Arabidopsis thaliana
  /note=“LLP genes identified in higher plants”
MRNNHSLRLQLWFRTLFTVGVVTLLMAKEIQQELEDGSRNDDLSYVASKRKVPRGPDPIHNRFLLLSRFILSLLTNPYPYLHICVLDVSV

SEQ ID NO: 94 - protein, 102 aa, Medicago
  /note=“LLP genes identified in higher plants”
MTYKYNITREKALHMDTNSNTSSSSFLFSRFFFRVLVVVGFVCFMVFSSLVSGGGTGKDKGVVVNHDAQLDFNYMSKRRVPNGPDPIHNRRAGNSGRPPGQT

SEQ ID NO: 95 - protein, 36 aa, Artificial Sequence
  Description of Artificial Sequence example for blast
EEAKILLGDSKETITNSTALESKRIIPTGPNPLHNR

SEQ ID NO: 96 - DNA, 1089 bp, Brassica napus
  promoter (1)..(1089): /note=“Promoter for BnLLP1”
acttgtgcgttaataatttggaacactataagcggagaagacataaaatggaatgccaaataggagtaccactgccatgtatgtttctgcattaatactgtgcccatactcacatttaattasgagtatgctgattttrcgcatgagttatcaaacgtgacagaacgttatagagacatgttagaaaagcacgaagcctgaaaaactgcaaaagtttacaacaaaaaaaactgataactacmattcttggcatttattgtcctttatttttgtaaataggtcttaaatacgtaataattttcttttctttttgacgaagatatgtcgcttaattgtgtgctaatgccgtcgttcatgatcgtctttcatgcatatggagtaactgaacatgacactgattcaaaaaaaaaaattaatttcttacgaagttttgaataatagtttttttaccacattaagtacactttaataaaaaaggaacacagtctacaaatagtttgagatgacattgactattatcatctaacttttattctgcataattgatttatcaaaaataattataatttatagtataaatgtagcctctaaatttagatttggcgatgataaccatgtcctaaacttacaacggaccagtaagcggtcacaaatctatcggaaaatttcgtccctatagcagaatgtttgggtttcctctttatttattattgtattcaatgataactggtcggtttactgttaccataaatgtgtccacaggctcacttaatgaggggtgatgcttatgaatcttcaaatcaaagacataatgaagaactttttcatagtgaaacgagataaagatcgaggaacatgcatgattccacataattacctaattatttttcagctaaagtatttcttttaatatgtaacatgcttcctttatttttagtgcaccgtagcttgtatataaaattttatccacacaaaytccttttgctccttttatcaaacgtcgctatttgtatcagtctacctatttgctttcatattgtatctcccgcagccaaacaaaaactttttttttgacaaagatagaacatg

SEQ ID NO: 97 - DNA, 1273 bp, Arabidopsis thaliana
  promoter (1)..(1273): /note=“Promoter + 5′ UTR + coding region from AtLLP1”
gaaagtcaagaaactgagagaaagagagatggtgtgaagttgaaaatgaggaaaataatccttttgggggaaaaagaagttaatgcgtttgattgaaattgaaagtggctagtaggaggaagtaagtaagcatgggttacattacaatttcaccttttcacttacctttaaacttcacttccaaaataattacacttttcatgtgctttgtttcaaaggtttctctgatactctcttcttcatataatagaacccatatataatgtactatatatatatatatatatatatatgaaaactctaattaaggtgttaaagttaattgggaatgcggaagaaccagaatatattcacatttcatggtaaattataagtttttcgtttattgtgaaagttcttatgggatggacatattatgatcatgtagttatatagattagttgcaaaatacaaatatcaatttgcatgattcatgttttttcgaaatgtgcattaacattcgcctctatacaaagaatggtgatgatagaattgtcataaaaagaagatggaccgctaagaggtcacaaatctattgcaattcgtcgtccataatgaatcgtaaatgtatttttcttagacctcatcaatgggtagcagaatatttgggttttattcattaagaaaaactatttattgtatacaattataacatgtcggctttactgttatcataaatgtgtccacaggccacttaatgaggctaaggcttatgaatcttcaaatcaaagacataatgaagaacctttttttacctatcgaaacgagaaaaagtggaacatgcatgatttccacataattacctaattattcttaagctatttcatttagtcaaatgcttcatttatttattttatatgcaccgtatagcttgtatataaatatatccattctaattctttttgcccttttatcaaacgtcgctatttataacaatccagcttttgctttcctccatattatatccccatcaaacaaacaaaaacaatatttgaccaaaaatagacaaatgaagataaagggtttgatgatattggcttcttctctcctgattcttgccttcattcatcagtcggaatcagcttcaatgcggagtttgctgatgaataatggatcatatgaagaagaagaacaagttctaaagtatgactccatgggaacgatcgctaactcttctgctttagactcaaaacgggtaattcccactggtccaaatccactccacaacaggtaa

SEQ ID NO: 98 - DNA, 1300 bp, Arabidopsis thaliana
  promoter (1)..(1300): /note=“Promoter + 5′ UTR + coding region from AtLLP11”
tctagagtgcaatactggcatggctttgctcacacaatcactgtgagaatcaatgctataaggacactatcctgaccatgtgtaagattctaaatgatataggagaaactgatatatagtaatgaaattgtggggccagatctgttcaaactttggctttactatgttagaaggcaagcttttctttcccaccaaggccaaaaggaagtttgcttgtgtcatggaaacaaactggctcaatcttccctagctgtcaatcagatatagattctttttttttcacagtttagatatgtttcttaatgtttttgctttagtttatcgtttaaaggttatgaagaacaagcatacatttcttgctgctaattggcttttggtagggagggaatacttaacaaggaagaaaaattgtaggctctagctccttttcacaggtttggttatcttacttgtgtattgttacattcttaattgattttgttattgtttaaaggaaacatccaagtgatttaagatagtggtaaggaaagtagtaggcttgctaatacactcagtcacaaaaggtacactgtcctctgcttttaaaatggcttaaaatatatgtagttaagggagcttgtgaggtccccaatggtttgctgcatgaaaattcacaaatcaaagcaaagaaaaaggtactcaacgggcccatatgttacataaagatatgttttttcttaactttcacttgctcttgcctcctctctctctctctacgtggacacctacgtatgtatattgttgctttgtgttgcttcatctttaaatagggccaatcacggatttgtcttccaaggtatataaaaacaacctaaatgcttgttttttgcttcccatttcgctttccccttttttgagcctcttctgtccaaagatatctctctctatttatgtgacagtcacttcaccaacatcatggatgttctgctcagtttattcttgggtttggttggtcagttgtttatgttaaacaggaagctgtagggacataggtttcagtatgggtggaaatggcattagagctttggttggagtgattgcatctttgggtttgattgtgtttcttcttgtcggtatcttagcaaactctgcaccaagtgttccatcatcagaaaatgtcaagactttgcggtttagtggtaaggatgtgaatctgtttcatgtaagcaagcgaaaagttcctaatggacctgatcctatccacaacaggttcttatcccttctctctcgaattttcaatcttttacttttgcttctttga

SEQ ID NO: 99 - DNA, 2050 bp, Arabidopsis thaliana
  promoter (1)..(2050): /note=“Promoter + 5′ UTR + coding region from AtLLP12”
ggtatcgaattcccgcctaaaattttgattttagcaagtgatcgagaaaggacagagatcgaagaaggttcgagagagcgaaaaagcgagactagttagggtttgagagtcttttgtgtctgatgaccaatgagagaggaattattctctgttttcttccgttggcttctctctcaatataatcaacaaattacagtcagcctttttttattattattcaagggctccgcgtgggcttcacgcgctccttcaacacggcccacttaaccagacaaatcaaggatcttccattagctaaaccgtattcgaaccgagtaatgaatctctctttaaccggtggtactcttatccaaacaaaaatatccacgactatttatatccgatttagattgttatctctttctatattttttatttacttgagtgaaaaacatgaattgttattctaaaaaaccattataagaccgaaaattatcaaaacataaataatgtaagttttcagtgttttgggttggtcggacaggtctaatccgatcgagacatgcctaacaatatcttctacaatttaagatgaaacatacgatgaatacatacgtgtatatttgctgatataacatcagagacaaagtgcttgacgagaacaaaatggaataatggtatttttggggatttgagtttgtatacctgctctctactatgtaaatgatgatttgtctgctgtaaaataaatgggaatataatatataaaaacgtgatgactgatgaggatatattgaggtacatatcacatatggtgtttcttgacaaatattggacgtaagtctttgttttttcttctataaactttcagcttgatttcttaaaaacatttgttgcacaagttgtaaatacgtttatcgaaggtagaagatgcatattggagaatcatatccatatatgtatcatgtatgtaatttagttcttgtgatactgttttctttattctctttcttaactcataataaagtaaaattaaaaaaaaaaaagtaatcttagaaagactaaaaaacgagaagcaaatatagatatacaacatttatataaaatggagaggaagaagattcaagtggtaagtggattttttcatgtgtgtgagtgagtgtaaatgtgggtgaagaaaaagggagtgaagacaacatcattttccccaaagaaagctaagtcttagaatcatcactccaattccctttttggccccttcttctctcttttctatttctttctacttttgacccccttcacataattaccttcctctatatgctttattatcacaccctcattactctctaaaactaaccactataagtatattattaaccacactaattactaccacttgacctagtaatcccagctaagacctttggaaatgattcaacacacaaaaaaaactttcgttaaaaattaatgatatatatatttacaaacctctatctagtttctcttctctttatttgtagaaagtggtggagaataagaaagggagagcaacaaacttttagaggaagtcataggttaaaagccccatagctttttcttccatcactttcattcctctcttctcattcttacagactttgagagagaatttatttaagagatcttgatcagatcttttatacttgcaaaaatcgattctaaatatataatctataccttatagagaaactcttcatatcttgaagattcctaagagatgggaaattactactctagaagaaaatctaggaaacacatcactacagttgccttgatcatccttcttcttcttttgtttctgtttctttacgctaaagcttcatcgtcttctcctaatatacatcatcactcaactcatggaagcttgaagaaatctggaaatttggatccaaagcttcatgatcttgactccaatgctgcgtcatcaagaggatcaaaatatactaattatgaaggtggtggtgaagatgtttttgaagatggcaagagaagggtcttcacaggtcctaatccattgcacaatagataa

SEQ ID NO: 100 - DNA, 1717 bp, Arabidopsis thaliana
  promoter (1)..(1717): /note=“Promoter + 5′ UTR + coding region from AtLLP5”
attcttttatcttcggacaaccgatttccaaaaagtgatacgtttgaatgattttactctcttttttcttcattcaaacgtatcactttttttttgtttagctatgtttaattaatcacatactatatcttataatatccacaattttatagttccttatatctgaaatactacaaaagcattttggaatcgcataataataatgatgatgggatctttgcataattctgacacgaagaggaagatgcgaaagaaggcaagaagaagagacgaaaccaaagagagaaagaaagaggaacaaaacaaacacaaaacggcaaagtgcagttaatgaaacgacaccctttttccaaactcatctctaacatacaaacatccaaaggaacaattttgtgttgtattgaacaaaaccaaacgtaatcactatttgcttacgtcacactatcttcagattttctcaagagacgcttacataataaaatatgaagttttctttgttggtatttgtaataattattttgtagttaaataactatgaaatcttaattgacaattcctttcacacaatgctactagttttaatatacagtctatgctcatagccatttacggtctaatgttttagactttagtcatgtatgtcaaaaatgataaacaaaaattattgaaaggaatggtggcacggtttttgtttagcataagatccaatgatccacattcaaacccattaaattcaaattttcttgtatatataactgtaccaagtcattacatcaataatgttccaaagtaaacctattctacatcatctatataaagctaactccaaaacgacaacaatttgaaattcataccaaaaatgggtaagtacgttggtgggtggtgcatttattgatttttgaaagttctgtttttcccgtacgtctaaagaatcaatatataaattgtttatatttgtatatgtaattttagatacagaatctaattataaagcaatgtacaatgtaataattcttgacaattccagaacaagagacaaaactattttaaattaatttttatatatgctcgtacagcttaaataatatactgtatatacacagttgtacaccaataatagtactataccactatttatcataattagatatgtgaaaatatgtactagtaatgaaacgaaaaaaaaactaaattaatcaattctctgatcttgtcttgtatttatatataaccattcacgtgggccattaaatttgcatgaaagccactcttcttctcacaaaacaaaacttcaatcttttgctctctctctctcaacttcagaaatagtcatagaagatagatctaacccagagaagacaagacagaaagaagagatataaaatcgatgattagggtttaatcaagaagacgactttatgttagggtttaggtttgggatatgactcatgctcgagaatggagaagctctttgactactacacttctaatggtgatcttgctttcttatatgctccatctcttctgtgtatattcacgggtaggggcaattcggatatttccagaaactccggcttcgggtaagagacaagaagaagatctaatgaagaagtacttcggcgccgggaaatttccaccggtggattcttttgtcggtaaagggatcagtgagagtaaaagaatagtaccaagttgtccggatcctttgcataactag

SEQ ID NO: 101 - protein, 91 aa, Arabidopsis thaliana
  /note=“Coding region AtLLP5”
MTHAREWRSSLTTTLLMVILLSYMLHLFCVYSRVGAIRIFPETPASGKRQEEDLMKKYFGAGKFPPVDSFVGKGISESKRIVPSCPDPLHN

SEQ ID NO: 102 - DNA, 1552 bp, Arabidopsis thaliana
  promoter (1)..(1552): /note=“Promoter + 5′ UTR + coding region from AtLLP2”
actactgtgttcacacgtcgactataatttagacctattgatacgatatgaattattgcaatcatctcctgcaaatataatagcatcgattacacatctttataatcaaagatttttattattttactttcataattccaaagattttttcataattaataagttgcaatcatgcctcctttttcgttccttgaattctaagaaacctcgatgcattagtttaattttctttacacggtttattacacattatgaattttcttttgaatatcaattattgaccactatagatttgatttaagtacgtattattactattttggagtaattaatattcgctttcatggtctttaagttttttaacgttatctgacttgatattttttttttgttggcttcaaactaagactctgattatttttatttagttgtttgttcttaaccaaaaagagattatgtttatctataataaacgtgaattactatttaaacataaaaattcaatgcaaatttaaatttcctttaaaagaatcaaaactagttcttaatgatttttttctaatactatacgaaatggtacttaacacatagataaataaatgaattcactaatagagtaatattgtacatccatattttattattattaataaatctgttttcagaattgcaaaatttctttctaataaaaaagataaacgcaaatatacacatcggtctaaaatcgaaatctctatgctagagaggagaactaattaatataaacatgtttaggtactaggttcaaaaatattatgaacctatttaagacttgcaagctcttggtaaaaagtaaaaacaaataatataccgatcataaaactgcaatctacgacagttttgactaccgccaaagctaacaaaatatcagccagtaggcaaaattttgacgcaaaaataagctggacagttttgtaaaatggtgtacatatgtcatatgataaagcatcttgcgttgaaaagacaaaatccaactctctcgcatctcttgagctttacattttgtcaactatatcatatataaatatagatgagtcctttcatactctcttctaaaccgtccaacaaaacctttttagtcttttttccaaaaacatacaaaacataaaaaaaaaacaagaatttagttttgtggcctattgattagcacacccatatgcttagaatttcttcatcatcttccatggccttgaaattttctcaaattctcttcatagttttatggctatctctcttctttctcctcctccaccacttgtactccctcaacttccgtcgtctctactctctaaacgcggtggagccatcattgttgaagcaacactaccgtagctatagattagtgagcagaaaagttctctccgacagattcgacttcacacctttccatagccgagataacagccgacataaccatcgttccggcgagcaatacgacggtgatgagatagaccctcgttacggagtggagaaacgccgtgttccctccggacccaatccgttgcaccattga

SEQ ID NO: 103 - DNA, 1383 bp, Arabidopsis thaliana
  promoter (1)..(1383): /note=“Promoter + 5′ UTR + coding region from AtLLP7”
ggagggacgggtttttgaatcaacattaataaaaaagtaaaatataattgatccactgtttcaatacgggttaaatatttaattattattttttaagaccactaatattaaacatatcaaatcatcctaatttagaaaaggttatataaaaccaaaaatgttatgtggtatgtataatgttactatatataaaattaaactataaaatataaatgtattagagaatgatacaatttgtaaaacttttatatgtaataaataattttcaaatttttaaaattactactttaaaaataaatcacgggatgggtaaagaaattacagaacggattttattttggaattgagttatatggtggatgtatttgaatcaatatttataaaattttaaaatattattaatatgctgttttagtaagggttaaaacttcagttttttaacaattgtctcatggattcgttgtatagcgttacttaataacaattataaactgtaaaatataaatattttataaaaataaaatttgcaagttttaatatatattaactttaaaaaataaattataccgcagtataccgcgggttaaaatctagtttatatatatttcacttaattttgtctatgttcactatacatatgtaaactcaagagtcaagactattcaattatatcatcatttcgcaaacttataataacaaattatataatatagtgataagtgaagttttgcttactatttatcaaagatttcatcttattttcaacaattctttcaatttgtaatgttatcagtaaaaaaattgtagtatataatctgctagttaataatacgaaaatatttgttattattgtacgaaattatcaattatgataattgtgtagtcatatcaatgtaatgaatgctgctgaccattctatttcttctctccttattcccctttgctcctccttacttttactaaaattccctcaactcttacgataagagcagagagaagaagaagaagaagaaagtaatcgtgatttgagagagagattcaaacatacttttttcgatatctatgttaattttatcttcacgatatgcaatgaaacgagatgttttaatcattgtaatctttacagtgttagtattaatcattatctcccgttcaagttcgatccaagctggaagattcatgactacaggaagaaatcgtaatttgagcgttgctcgatcactatactacaagaatcatcacaaggttgtgatcacggagatgtcaaattttaataaagttagacgaagatcgtctagatttcggagaaagacggacggcgacgaggaagaagaagagaaacgatctatccccacaggtccaaatcctttgcacaacaagtag

(19) United States
(12) Patent Application Publication
(10) Pub. No.: US 2004/0110121 A1
(43) Pub. Date: Jun. 10, 2004

(54) Plant-signalling ligand like proteins

(76) Inventors: Chun-Ming Liu, Wageningen (NL); Johannes Hubertus G. Cordewener, Wageningen (NL); Martijn Adrianus Fiers, Ede (NL); Ronny Viktor L. Joosen, Renkum (NL); Apolonia Helena M. van der Geest, Wageningen (NL)

Correspondence Address: HOFFMANN & BARON, LLP, 6900 JERICHO TURNPIKE, SYOSSET, NY, 11791, US

(21) Appl. No.: 10/311,174

(22) PCT Filed: Jun. 15, 2001
(86) PCT No.: PCT/NL01/00452

(30) Foreign Application Priority Data
Jun. 16, 2000 (EP) 00202118.6

(51) Int. Cl. C12Q 1/00

(52) U.S. Cl. 435/4

(57) ABSTRACT

The invention relates to the field of plant growth and development, more in particular to the communication between plant cells influencing architectural or phenotypical characteristics such as their rate and pattern of division, orientation of elongation, organogenesis or differentiation patterns. The invention provides a method for modulating plant phenotype or architecture, such as by modulating or changing plant growth, it's development or defense responses, by modulating its rate or pattern of cell division, orientation of elongation, organogenesis or differentiation patterns, comprising providing a plant or plant material with recombinant ligand-like protein (LLP) or a functional fragment thereof, said protein or fragment at least comprising an LLP box as provided by the invention comprising an amino acid motif XRXXXXGXXXXHX.

The invention relates to the field of plant growth and development, more in particular to the communication between plant cells influencing architectural or phenotypical characteristics such as their rate and pattern of division, orientation of elongation, organogenesis or differentiation patterns in response to developmental or environmental stimuli.

The fusion of egg and sperm produces a zygote (also called fertilized egg). The single-cell zygote goes through a successive cell division and expansion process to generate a massive amount of cells that contribute to the body of a plant which can vary from a giant tree to a small grass, or from a potato to a peanut. Plant cell divisions are highly regulated, which give each plant or part thereof a specific shape or architecture.

There is no doubt that a precise developmental mechanism is present in plant cells to regulate its rate and pattern of division, orientation of elongation, organogenesis and differentiation. Such a developmental program is controlled genetically by genes in the nuclei and to a lesser extent in the chloroplast and mitochondria. During the last fifteen years, molecular genetic approaches have been used extensively to dissect such developmental pathways, especially in model organisms such as Arabidopsis, Petunia, maize and Antirrinum.

These studies have for example led to the identification of genes regulating flower development (Yanofsky et al, 1990; Mandel et al, 1992; Jukufu et al, 1994; Weigel et al, 1992), embryogenesis (Lotan et al, 1998), meristem identity (Long et al, 1996), light (COP1) and hormone signal transduction (PIN, ETR, BRI, brassicasteroid, GAI, Peng), etc. The products of a large number of these genes turn out to be transcription factors which can bind to the promoter regions of downstream genes to initiate or suppress developmental pathways. Transcription factors include Myb, MADS, KNOTTED and AP2, etc. Mutation in one of these genes often leads to homeotic conversion from one organ to another. The expression of these different genes defines organ identity and the fate of differentiation of certain cell types.

Different from animal development, plant cells do not migrate during development and fate in a plant cell is less determined than the fate of an animal cell. Therefore, some perturbations that would cause abnormal growth and development in animals fail to affect normal plant morphogenesis. For example, over-expression of the cell cycle gene cyc1At increases the mass of the root but not the structure and morphology, and the tangled mutation in maize that failed to execute normal longitudinal cell divisions, is relatively normal in morphology. In plants, each cell needs to communicate and co-ordinate with its surrounding cells. Although plant cell division follows certain patterns with a traceable fate map, laser ablation experiments have revealed that when one or more cells are killed, those cells next to it are able to replace such cells, and even cells originating from a different layer which have different developmental origin have this capability (van den Berg et al, 1995).

Therefore, position itself is a very important signal for plant development (Hake and Char, 1997). Now the questions arises how position signals are accessed, how they are transferred between cells and how plant cells can sense such signals. Small signal molecules such as auxin and ethylene can diffuse through cells walls, while receptor kinases contain extracellular domains for ligand binding (Fletcher and Meyerowitz, 2000). Some proteins, such as transcription factors and viral movement proteins, can travel between cells through plasmodesmata (Lucas et al 1995; Citovsky and Zambryski, 1991).

Another way of cell-cell communication is through receptor-like kinases. There are four classes of receptor like kinases in Arabidopsis now known, based on their N-terminal extra-cellular domain sequences. Group representatives are:

The leucine-rich repeat (LRR) group, which is the largest group. LRRs occurs in numerous eukaryotic proteins and are thought to be involved in protein-protein interactions. LRR is also present in several mammalian receptors for protein/peptide messages including nerve growth factor receptor. This group includes ERECTA, CLV1, CLV2 and BRI1 (BRASSINOSTEROID-INSENSITIVE1). BRI1 is most likely the receptor of brassinosteroid. The expression pattern of currently known receptor kinases can be used to refine the function of LRRs.

The S-domain group, which have extra-cellular domains related to the S-locus glycoprotein of Brassica species involved in self-incompatibility response. Three S-domain RLK are found in arabidopsis, but they are not involved in self-incompatibility since they are expressed in inappropriate locations, and the species does not display self-incompatibility.

The lectin-like domain group, related to legume lectins. They may bind to oligosaccharides such as elicitors derived from the breakdown of cell walls of pathogen or plant during fungal infection.

EGF repeat receptor, represented in Arabidopsis by WAK1 and WAK4. Extracellular domain is related to mammalian epidermal growth factor.

Without ligand, the receptor-like kinases usually are present as a monomer in the membrane. The binding of a ligand to their extracellular domains leads to the formation of homo- or hetero-oligomers, usually dimers, to initiate a down-stream signal tranduction pathway by protein phosphorylation. Such a signal transduction pathway has been studied extensively in animals and yeast. Since the first plant protein kinase was reported in 1989 (Lawton, et al, 1989), more than 500 of them have been identified in plants and 175 in Arabidopsis thaliana alone (Hardie, 1999). Most of these protein kinases are involved in intracellular signal transduction (calcium-depedent protein kinases), stress response (leucine-rich repeat receptor kinases) and cell cycle regulation (cyclin-depedent kinases). Some protein kinases, for example, members of the two-component histidine/aspartate kinase family, are involved in hormone signal transduction, for instance, ethylene and cytokinin (Chang and Meyerowitz, 1995; Kakimoto, 1996). The recently identified ERECTA, BRI1, CLAVATA1,CLAVATA2 and HAESA are examples of receptor-like kinases which may be involved in cell-cell communication (Ku et al, 1996; Clark et al, 1997; Jeong et al, 1999; Jinn et al, 2000). Based on the outcome of genomic sequencing of Arabidopsis, it is expected that there are more than 100 receptor-like kinases in the Arabidopsis genome (Fletcher and Meyerowitz, 2000). Mutation of CLAVATA1, CLAVATA2 and CLAVATA3 showed almost identical phenotypes, enlarged central domain of meristems and increased floral organ numbers (Leyser and Furner, 1992). CLAVATA3, which was cloned recently, encodes a small predicted extracellular protein with no significant homology to any known plant and animal proteins (Fletcher et al, 1999). Based on phenotypic and biochemical analysis, CLAVATA1 and CLAVATA3 are believed to be different components of a signal transaction pathway, although direct proof for such interaction is not yet available (Clark et al, 1995; Trotochaud et al, 1999). Based on these results, it is very likely that CLAVATA3 is a ligand protein identified from higher plants, which interacts with the CLAVATA1 receptor kinase.

The invention provides a method for modulating plant phenotype or architecture, such as by affecting or changing plant growth, its development or its defence responses against external stimuli or disease, by modulating its rate or pattern of cell division, orientation of elongation, organogenesis or differentiation patterns, comprising providing a plant or plant material with recombinant ligand-like protein (LLP) or a functional fragment thereof, said protein or fragment at least comprising an LLP boxmotif as provided by the invention comprising an approximate amino acid motif XRXXGXXXH or (1)R(4)G(4)H(1). The method provided herein essentially comprises modulating plant phenotype by providing for ligand-interaction between a LLP box motif present on a protein, and its corresponding receptor or binding site. Said LLP protein or functional fragment thereof at least comprising said LLP box motif, when bound, than provides for a further step in a cascade of steps in plant development, and by using a modified or recombinant LLP protein, it is possible to generate novel cascades and thus novel phenotypic manifestations in a plant.

A preferred amino acid LLP box motif to select for comprises K R X X X X G X X P X H X. In particular, a preferred box comprises a consensus sequence showing at least 80% homology with a preferred consensus sequence K R X (V/I) (P/H) (S/T) G (P/S) (N/D) (P/H) (L/I) H (H/N) (bold amino acids typically are most conserved). Such LLP box preferably starts with KR or ends with PLAN or has no more than 10 amino acids C terminal of the box. Furthermore, it is observed that the majority of the LLP motifs in FIG. 13 have 3 prolines out of 13 aa in the LLP box, giving them a very unique 3D structure that is required for their function. Some members from other species than arabidopsis have only 2 of the 3 P residues (the middle P is an S), and only one LLP (LLP6) has only 1 of the P residues. Generally speaking, the LLP box starts with 2 very basic amino acids (pK 10 or 12), has a hydrophobic amino acid in the fourth position, followed by a proline (introduces bend or kink), and than two small amino acids (one with a hydroxyl group and one glycine), another proline (or serine), aspartate or asparagine, another proline and three amino acids with bulky side chains. This sequence produces a recognizable 3D conformation that is involved in receptor ligand interaction. The LLP box is an amino acid motif that is shared among all the LLP genes and is important for their biological function in signalling, for example by mediating interactions with the receptor, folding of the ligand into the proper conformation, and/or by binding to other cellular components that regulate turnover after relay of the signal, Phenotypic responses include stress-mediated, hormone-mediated and disease-mediated responses, which have effects on plant shape, size, growth rate, reproductive ability (flowering, gamete and seed production), metabolism, and root and shoot development. In a preferred embodiment, a method for modulating plant phenotype is provided comprising providing a plant with a recombinant LLP protein or functional fragment thereof. Common features of the LLP proteins include their size, the presence of a signal peptide, and the conserved LLP box. These features all contribute to the role of the LLP proteins in signaling cells to alter their fate, thus allowing for example to modulate plant phenotype by regulating the level and location of LLP gene expression. When present, the sign peptide aids in the localization of the active LLP proteins and for example functions to direct the recombinant LLP protein to the extracellular space, where it can interact with the appropriate receptor complex to convey a signal to the receiving cell. The LLP box is a most critical feature for such interaction, in that it is conserved among the LLP class proteins, defining a common recognition domain for recognition of the appropriate subclass of plant receptor kinases, being provided with the right configuration needed for the specific receptor complex recognition. The non-conserved parts of the LLP proteins (e.g. outside the LLP-box area) provide the necessary additional specificity in order to convey different types of signals to the specific receptor complex it interacts with. Cells expressing the appropriate receptor complex (the signal receiving cells), interacting with the recombinant LLP proteins, respond by altering their fate, resulting in a phenotypic change in the plant. Thus, modification of the expression, location, and structural-composition of LLP recombinant nucleic acid allows modulation of plant phenotype.

The invention herewith provides an isolated or recombinant ligand-like protein (LLP) or functional fragment thereof from a plant, for example a plant such as Brassica napus (BnLLP1, otherwise known as DD3-12) or Arabidopsis thaliana (LLP1At), and its use to manipulate or influence plant architecture or modulate phenotype. LLP nucleic acid as provided herein in general encode ligands or functional fragments thereof that interact with receptor kinases which bring about the required phenotype response in plant tissues.

These phenotype responses also include alterations of cell fate, stress-mediated, hormone-mediated and disease-mediated responses. The invention thus provides a group of ligand-like proteins (LLPs) or functional fragments thereof with similar peptide structure and a conserved domain relatively close to their C-terminal, such as for example seen in LLP1, which are used to manipulate plant growth, development and defence response, and provides isolated and/or recombinant nucleic acid encoding said ligand-like proteins (LLP's) or functional fragments thereof

Altered nucleic acid sequences of this invention include deletions, insertions, substitutions of different nucleotides resulting in the polynucleotides that encode the same or are functionally equivalent. Deliberate amino acid substitution may be made on the basis of similarity in polarity, charge, solubility, hydrophobicity, and/or the amphipathetic nature of the residues as long as the biological activity of the polypeptide is retained. Included in the scope of the present invention are alleles of the polypeptides. As used herein, an ‘allele’ or ‘allelic sequence’ is an alternative form of the polypeptides described above. A ‘functional fragment’ as defined herein may b an allelic variant. Alleles result from a mutation, eg a change in the nucleic acid sequence, and generally produce altered mRNA or polypeptide whose structure or function may or may not be altered. Any given polypeptide may have none, or more allelic forms. Common allelic changes that give rise to alleles are generally ascribed to natural deletions, additions or substitutions of amino acids. Each of these types of changes may occur alone, or in combination with the others, one or more times in a given sequence.

It is envisaged that the polynucleotide sequence of the present invention can be used as probes for the isolation of similar sequences from other genomes (e.g. corn, rice, canola, soyabean, cotton etc). By using as a probe the gene sequence(s) of the present invention, it is possible to obtain comparable gene sequences. One aspect of the invention is to provide for hybridisation or PCR probes which are capable of detecting polynucleotide sequences, including genomic sequence(s), encoding the polypeptides of the invention, or closely related molecules. The specificity of the probe [whether it is made from a highly specific region, eg 10 unique nucleotides in the 5′ regulatory region, or the nucleic acid sequence of the LLP box motif or a less specific region e.g. in the 3′ region], and the stringency of the hybridisation or amplification (maximal, high, intermediate, low) will determine whether the probe identifies only naturally occurring sequence(s) encoding the polypeptide, allele's or related sequences. Probes may also be used for the detection of related sequences and preferably contain at least 50% of any of the nucleotides from any one of the LLP gene encoding sequences according to the present invention.

The LLP nucleic acids or functional fragments thereof as provided herein can function in quite diverse biological pathways, for example in: manipulating plant architecture, both of shoots and roots, manipulating embryo-endosperm interactions, male sterility, flower timing and organ identity, meristem activity, apoptosis (eg. suspensor vs embryo), stress (biotic and abiotic) response, senescence, leaf and fruit dropping, nutrition uptake from roots. Furthermore, they can be used in “regeneration”. “Regeneration” used as a general term for many possible applications of the LLP genes, such as competence, outgrowth, root formation, organogenesis, differentiation, vegetative development, shoot apical meristems, inflorescent meristem development, axillary bud formation and activation, or other processes where cell-cell communication or defining the boundaries of organs play a role.

The invention also provides isolated and/or recombinant nucleic acids additionally comprising promoter sequences that are functionally linked or physically adjacent to the nucleic acid coding region of LLP1 and other LLPs or functional fragments thereof as mentioned herein, which act as regulating elements in plant cells for developmentally regulating tissue or cell-specific expression. The definition ‘promoter’ is intended as a nucleotide sequence sufficient to direct transcription. Also included are those promoter elements which are sufficient to render tissue-specific gene expression; such elements may be located in the 5′ or 3′ regions of the native gene. In the case of plant expression vectors, the expression of a sequence(s) of the invention may also be driven by a number of previously defined promoters, including inducible and developmentally regulated promoters. The invention further provides the use of the individual promoters of the polynucleotide sequence(s) of the present invention for this purpose [for example BnLLP1 promoter (FIG. 16)].

The definition ‘host cell’ refers to a cell in which an foreign process is executed by bio-interaction, irrespective of the cell belongs to a unicellular, multi-cellular, a differentiated organism or to an artificial cell, cell culture or protoplast. The definition ‘host cell’ in the context of this invention is to also encompass the definition ‘plant cell’.

‘Plant cell’ by definition is meant by any self-propagating cell bounded by a semi permeable membrane and containing one or more plastids. Such a cell often requires a cell wall if further propagation is required. ‘Plant cell’, as used herein, includes without limitation, seeds, suspension cultures, embryos, meristematic regions, callous tissues, protoplasts, leaves, roots, shoots, gametophytes, sporophytes, pollen and microspores.

More preferred LLPs according to the invention also comprise a signal peptide at their N-terminals. The invention provides a method for selecting plant starting material or plants or their progenies for having a distinct LLP motif within one or more LLP genes. Such selection allows the detection of plants having a desired phenotype, by for example selecting plant (tissue) culture starting material, such as callus material or plants cells, having a desired LLP genotype. Selection can be performed using nucleic acid detection methods known in the art, such as polymerase chain reaction (PCR) or by hybridisation, using LLP specific probes or primers herewith provided. Additionally, this invention also provides plants or plant material transformed with the nucleic acid sequences encoding the proteinaceous substances[protein, (poly)peptides and (post-translational) modifications thereof] as provided herein (LLP1 and other LLPs). Such plants have in general altered phenotypes. In short, the present invention, provides a new class of ligand-like proteins (LLPs) which are small proteins with a conserved LLP boxmotif relatively close to their C-terminals and a signal peptide at their N-terminals. Such ligand-like proteins may also be recombinant proteins of a chimeric nature, or even be truly synthetic, in that they are derived by conventional peptide synthesis techniques. A ligand-like protein comprising said box motif as provided herein is useful for targeting a compound or recombinant or synthetic (poly)peptide provided with said box motif to a receptor where said compund or polypeptide can modulate signal transduction and interfere with communication between plant cells; thereby influencing architectural or phenotypical characteristics such as the rate and pattern of division, orientation of elongation, organogenesis or differentiation patterns in response to developmental or environmental stimuli. Such targeting is also useful for targeted delivery of a compound (provided with the box motif) to the near vicinity of said receptor

The invention furthermore provides a recombinant nucleic acid encoding a ligand-like protein or functional fragment thereof at least comprising an LLP box motif or peptide comprising an amino acid motif XRXXXXGXXXXHX, or a nucleic acid, such as anti-sense RNA, hybridising therewith. In one embodiment, the invention provides an LLP nucleic acid as shown in FIG. 3. Over-expression of the LLP gene results in changes in plant architecture, such as male sterility or deviant root development FIGS. 9-11). The invention also provides antisense LLP nucleic acid, primers or probes, be it of DNA, RNA or (peptide nucleic acid) PNA nature, hybridising with a nucleic acid as provided by the invention. Also provided is a nucleic acid according to the invention additionally provided with or comprising a promoter operably linked to a modified LLP nucleic acid. Such sequence can direct gene expression in axillary buds, floral organ primordium, stigma, and root-hair region and in the endosperm of mature and germinating seeds. Such a promoter is used to drive cell- or tissue-specific expression of a gene-of-interest. The invention furthermore provides a vector or host cell comprising a nucleic acid according to the invention, and a plant or plant material such as callus material or a plant cell provided or transformed with such a nucleic acid or vector.

The present invention is also related to the identification of a set of novel ligand-like proteins (LLP) that are structurally similar to LLP1. These proteins preferably have 50 or 60 or more amino acids, preferably have 75 or more amino acids, preferably have 85 or more amino acids, and preferably have no more than 250, even more preferred no more than 150 amino acids, and more preferably have no more than 120 amino acids; preferably they have a signal peptide at their N-terminals, said signal peptide preferably having a length of between 15 to 32 amino acids, as predicted by SignalP programme.

These proteinaceous substances have a conserved LLP boxmotif at their C-terminals, comprising amino acids XRXXXXGXXXXHX. Amino acids are herein given in the one-letter code, X stands for any naturally occurring amino acid. This LLP motif is preferably 55% or more, preferably 60% or more, more preferably 70% or more, more preferably 80% or more and most preferably 90% or more homologous to the LLP boxmotif or peptide as provided for Brassica napus (KRIIPTGPNPLHN; LLP boxmotif). Typical examples of an LLP boxmotif or peptide found in plants such as Arabidopsis are KRLVPSGPNPLHN, KLVPSGPNPLHH, KRRVPSGPNPLHN, KRRVPSGPNPLHH, KRLVHSGPNPLHN, KRVIPSGPNPLHN, KRKVPSGPNPLHN, KRSIPSGPNPLHN, KRKVPNGPNPLHN, KRKVPRGPNPLHN, KRSIPTGPNPLHN, ERLVPSGPNPLHN, ERLVPSGPNPLHH, ARLVPSGPNPLHN, ARLVPKGPNPLHN, KRVVPSGPNPLHN, KRVVHTGPNPLHN, KRRVPSGPNPLHN, KRRVFSGPNPLHN, KRKVPKGPNPLHN, KRKVKSGPNSLHN, KRLSPGGPNPLHN, MRLVPSGPNPLHN, or variations of these wherein singular amino acids are replaced by like amino acids (e.g. basic by basic, bulky by bulky, or acid by acid) or wherein for example the NPLH sub-motif within an LLP boxmotif or peptide is replaced by DPLH, NPRH or DPRH. Proteins comprising an LLP boxmotif or peptide can easily be found (or mined in databases) by e.g. BLAST searches using an LLP boxmotif, performed on polypeptide sequences generated with recombinant techniques well known in the art. Variation should preferably not functionally affect the LLP boxmotif in the (1)R(4)G(4)H(1) position.

In general the LLP [BnLLP1, LLP1at, and LLP homologs from other sources] have limited sequence homology to CLV3 proteins. CLV3 is a gene which functions as a regulator for the central zone of the apical meristem, possibly interacting with CLV1 receptor kinase although direct proof is still lacking (Fletcher et al, 1999). The LLP1 and other LLPs generally differ from CLV3 in at least one of three aspects: 1) very low homology, 21.1% identity in the overall protein sequences and homology in the LLP boxmotif is 54%; 2) no KR but instead LR is present; 3) preferred LLPs have the LLP boxmotif close to the end of the C-terminus of the protein, whereas CLV3 has a much longer C-terminal span. The length of the terminal peptide at the C-terminus of the LLP boxmotif should preferably be no more than 10 amino acids, more preferably no more than 5 amino acids and most preferably from 0 to 2 amino acids.

The invention provides also a recombinant nucleic acid comprising a promoter operably or functionally linked to LLP nucleic acids derived from different or heterologous plant species. Such sequences can direct gene expression in meristems, seeds or responding to abiotic and/or biotic stresses. Therefore, such a promoter is used to drive cell-, tissue-specific, stress-related expression of gene-of-interests.

The invention also provides a method for producing a plant having at least one or more cells transformed by LLPs nucleic acids, either by ectopic expression, misplaced-expression, over-expression, co-suppression or dominant-negative mutation. Such transformed or transgenic plants comprising a recombinant nucleic acid encoding a polypeptide with LLP-motif are also provided herewith

Down-regulation of these genes by anti-sense approaches or over-expression of one or more of such genes in plants will lead to changes in plant growth, development and defense response. The invention is also related to the identification of receptor kinases which bind to ligand-like proteins like LLP1 and other LLPs. Such receptor kinases are generally membrane associated proteins with an extracellular domain and an intracellular domain, which can now be identified by reacting with a ligand-like protein or functional fragment thereof as provided herewith.

### DETAILED DESCRIPTION

### EXAMPLE 1

### Cloning of BnLLP1 Gene (DD3-12) from Brassica napus L

Isolated microspores of Brassica napus cv. Topas at a stage around the first pollen mitosis were cultured either at 32° C. or at 18° C. The higher culture temperature leads to the formation of embryos and the lower culture temperature leads to pollen maturation (FIG. 1). Samples were collected at various days after initiation of the cultures and total RNA was prepared according to the procedure described in materials and methods. mRNA differential display RT-PCR (DDRT-PCR, Liang and Pardee, 1992) was used to isolate cDNA clones which appeared specifically under embryogenic conditions (32° C.). The DDRT-PCR gel of FIG. 2 shows a PCR fragment (named BnLLP1, indicated by an arrow) that was found in the samples of microspores cultured for 10 days (globular to heart shaped embryos) and 16 days (heart to torpedo stage embryos) at 32° C. (embryogenic development), but not in samples of freshly isolated microspores (t=0), microspores cultured at 18° C. (gametophytic development) or in leaf. This BnLLP1 PCR fragment was isolated from the gel and sequenced after re-amplification and cloning. Comparison with DNA sequences in NCBI GenBank revealed no significant sequence homology with known genes.

A cDNA library prepared from globular to heart staged embryos was screened in order to clone the full length cDNA of BnLLP1. This has led to the identification of a full length BnLLP1 cDNA (otherwise known as DD3-12), as shown in SEQ ID No.1 (FIG. 3). Analysis of this putative protein using SignalP programs (http://www.cbs.dtu.dk/services/SignalP/) indicated that this protein has a 23 amino acid hydrophobic transit peptide. Such a signal peptide will be removed during the transfer from inside the cell to outside. It is therefore expected that the final product of this peptide has only 51 amino acids. Proteins with such characteristics are normally working as a ligand protein interacting with one or several receptor kinases in the membrane of surrounding cells for signal transduction between cells (Jennifer and Meyerowitz, 1999).

### EXAMPLE 2

### Expression of the BnLLP1 Gene

The expression pattern of BnLLP1 as determined by Northern blot analysis is shown in FIG. 4. A high level of transcript was found in microspore embryos of a 10 days 32° C. culture (globular to heart shaped embryos). No signal was detected in root and leaf tissue, but a faint signal appeared in a mixture of flower buds of various developmental stages FIG. 4). Separate sampling of RNA from younger buds (1-5 mm), older buds (5-8 mm) and open flowers revealed that the highest level of BnLLP1 transcript can be found in the youngest flower buds. Within a flower a clear signal was found in pistels, but not in anthers and petals.

A BnLLP1 promoter::GUS fusion was constructed and transferred to Arabidopsis using a “floral dip” method (ref) to determine the expression pattern of BnLLP1 in a close relative of Brassica—Arabidopsis thaliana. Transgenic seedlings were selected on plates containing Kanamycin (FIG. 6). The GUS signal was first detected in the upper part of the embryos at later globular stage. At the heart-shape stage the GUS expression is restricted to the top but slightly close to the abaxial side of the cotyledons. Further development of the embryo led to the change of the expression of BnLLP1 to a narrow tier of cells at the edge of the cotyledon (see FIG. 5). At the cotyledon stage the BnLLP1 expression was localized to a ring-shaped region at the base of each cotyledon, but not in the embryo including the root and apical meristems.

Interestingly during seed maturation, the expression of the BnLLP1 was seen in the remnant of the endosperm, a single layer of cells located between the testa and the embryo. The GUS expression was continued till the first few days of seed germination.

During post-embryonic development, the expression of BnLLP1 is restricted to axillary buds, flower buds and mature roots, not in leaf, flower, or vegetative meristems. In Arabidopsis each axillary bud will normally form one new inflorescence which has 2-5 cauline leaves and indefinite number of flowers. As soon as flower starts to form, no cauline leaf will be produced. Generally, only one inflorescence is produced from each axillary bud. In the axillary buds, the expression is restricted to leaf primordia and moved quickly to the abaxial side of the peteols when leafs are expanding (FIG. 8C). In the flower buds, the BnLLP1 expression was first seen in the stage 3 flower buds at a periphery of the flower primordia indicating the positions where sepals are forming. In a stage 5 flower, the BnLLP1 is no more expressed in the sepals which has already formed, it is in a region between sepal and carpel primordia, where petals and stamens are going to be formed. In a stage 7 flower, when stamens are forming the BnLLP1 expression is seen only at the top of the carpel where stigma is forming. The expression of BnLLP1 was switched off completely before the flower opens.

In roots, the expression of BnLLP1 started after root hairs are formed, 6-7 days after germination (FIG. 7). No expression can be seen in the hypocotyl and the expression margin between hypocotyl and root are very sharp. Within the root, BnLLP1 expression was excluded from the epidermal layer on which the root hairs will be formed. The BnLLP1 expression was gradually switched off in the cortex and the ground tissue to the vascular boundles, and later to the pericycle and then off completely when the root hair starts to degenerate. Apparently the BnLLP1 expression is associated with mature roots with well developed root hair. This is the region where root functions dominantly for nutrient intake from soil. No BnLLP1 expression was seen during lateral root induction, nor the old root which functions as a supporting and transporting organ.

### EXAMPLE 3

### The Phenotype of BnLLP1 Over-expression

Doubled enhanced 35S promoter was used to drive the over-expression of full length of BnLLP1 gene (otherwise known as DD3-12) in Arabidopsis. The transformation was carried out using the floral dip method mentioned previously. Three independent transformants with almost identical phenotype were obtained from 2 transformation experiments. These plants are slow growing and late flowering, bolting only 45-50 days after seeds were planted instead of 20 days in the WT. A dramatic phenotype of these BnLLP1 over-expression plants is their changes in branching patterns (FIG. 9). Instead of one branch was formed at each axillary buds, these plants normally have 2, 3, 4, 5, and even 7 inflorescence produced at the axillary position of cauline leaves. The formation of new inflorescence are gradual, starts with one branch and new ones are formed during the growth of the plants. These BnLLP1 over-expression plants are male sterile, no viable pollen can be produced in the flower. The anther also stays very small, in a triangle shape. Another change in the BnLLP1 over-expression plants is the formation of pin-shaped carpel in 80% of the flowers (FIG. 10). These pin-shaped carpels are slender structure without formation of ovules inside. A stigma-like structure can be observed at the top of the carpel indication that the expression of BnLLP1 may function as a signal cue for ovule induction, rather than the formation of the stigmatic tissue. Those 20% flowers with normal pistil are fertile if pollinated with pollen from wildtype plants. A careful cytological analysis has showed that the BnLLP1 ever-expression plants have defects in building up vascular strands, especially in flowers (FIG. 11).

### EXAMPLE 4

### The Identification of Other LLPs in Arabidopsis

General BLAST or BLASTP searches through NCBI and Arabidopsis database using either BnLLP1 cDNA sequence or protein sequence showed no significant homology with any known cDNA or protein sequences. Our first attempt in searching existing protein databases SWISSPROT using BLASTP in NCBI and the TAIR (an Arabidopsis database) GenBanks have showed no significant matching sequences. However, based on the sequence alignment between BnLLP1 and LLP1At proteins we found that the C-terminals of these two proteins are highly conserved, which might be associated with the important function of these genes (FIGS. 12 & 13). We then used the C-terminal sequence to do the database search with a modification of several parameters (FIG. 14). Instead of using protein-protein homologous search, we used the C-terminal peptide sequences to search all nucleotide sequences in NCBI and TAIR databases with 6-frame translation. This will allow us to access all the possible sequences in the databases. Such a search has led us to the identification of a group of proteins with highly conserved C-terminal boxes, thirteen of them are from Arabidopsis (FIG. 15), one from cotton and one from soyabean. This is a boxmotif which has never been identified before. We termed it as LLP boxmotif. Interestingly, all these proteins are very small, ranging from 60 to 120 amino acids (see for examples FIGS. 17-22). Additional members have been identified which belong to the LLP family using this search criteria (FIG. 23).

All of these proteins with a LLP boxmotif have an N-terminal signal peptide with 15 to 32 amino acids, as indicated by SignalP analysis (ttp://www.cbs.dtu.dk/services/SignalP/). Such signal peptides control the entry of virtually all proteins to the secretory pathway to outside of the cells. The signal peptide will be cleaved off while the protein is translocated through the membrane. The common features of these signal peptides are a positively charged n-region, followed by a hydrophobic h-region and a neutral but polar c-region. A (−3,−1) rule states that the residue at the position −3 and −1 (relative to the cleavage site) must be small and neutral for cleavage to occur correctly (Nielsen et al, 1997).

Based on these three common features, it becomes apparent that LLP is a new class of protein. They may function as ligands to interact with receptor kinases in the neighboring cells for cell-cell communication.

Since LLP genes encode ligands that are able to interact with membrane bound receptor kinases in order to induce a signal transduction cascade, it is possible to make use of this interaction for other purposes. Redesigning a LLP ligand in such a way that a stable non-productive interaction occurs between it and its receptor will result in a competition between the modified and the wild-type ligand for receptor binding. Substituting certain amino acids in the receptor interaction domain will create a stable, non-functional ligand that will occupy the receptor binding sites, resulting in a dominant negative mutant phenotype where the signal transduction cascade is blocked. This can be used to alter plant architecture.

The interacting domains of the ligand and receptor can also be used in a different context, by linking them to other proteins that normally would not interact. In this way new protein-protein interactions can be created in planta.

Several known proteins have certain similarities to the LLP proteins. These are CLAVATA3 protein from arabidopsis and ESR protein from maize. They are also small proteins with a signal peptide at their N-terminals. These proteins showed certain similarities with LLP in the LLP boxmotif as well, certainly the similarity is lower. The most distinct differences are the location of the LLP boxmotif. A somewhat alike box in CLAVATA3 and ESR proteins is located much further away from the C-terminal end than LLP.

As mentioned above, most LLP proteins shown here have the LLP boxmotif 0-3 amino acids away from their C-terminal ends. One animal protein, a putative RHO/RAC guanine nucleotide exchange factor (RHO/RAC GEF) isolated from mouse (accession No. P52734), also showed certain homology with LLP proteins in the LLP boxmotif. In this case, however, the assumed LLP boxmotif is much further away from the C-terminal and even closer to the N-terminal (Pasteris, et al, 1995). This is the first time that the LLP sequence motif has been identified in any organism. In plants we find this motif generally associated with small extracellular ligand-like proteins.

### EXAMPLE 5

### Finding Novel LLP Genes and Peptides

Given the conserved nature of the LLP motifbox, the public sequence databases were searched for sequences or putative ORFs that encode proteins containing a K R X (V/I) (P/H) (S/T) G (P/S) (N/D) (P/H) (L/I) (H/N) domain. Many Arabidopsis LLP box-containing ORFs were identified. Many of these were not yet annotated in the database. Extrapolating from the LLP box, the start and stop codons of the ORF were identified. The size of the predicted proteins encoded by these ORFs ranged from 100 to 250 amino acids, and all had a high probability of encoding a signal peptide at their N terminus. The following Arabidopsis ORFs belong to the LLP family based on the size of the predicted protein, the likelyhood of a amino terminal signal peptide, and the presence of the LLP box:

Located on chromosome 1, on BAG clone F23o10 accession AC018364 (34983 until 34660) from Arabidopsis thaliana

Located on chromosome 1, on BAC clone F2OP5 accession AC002062 (108432 until 108788) from Arabidopsis thaliana

Located on chromosome 1, on BAG clone T1K7 accession AC013427 (8397 until 8769) from Arabidopsis thaliana

Located on chromosome 2, on BAG clone F2I9 (section 4 of 255 of chromosome 2) accession AC006069 (55097 until 55786) from Arabidopsis thaliana

Located on chromosome 1, on BAG clone T7A14 accession AC005322 (16956 until 17207) from Arabidopsis thaliana

Located on chromosome 1, on BAG clone F12A21 accession AC008113 (31668 until 31928) from Arabidopsis thaliana

Located on chromosome 1, on BAC clone F15H11 accession AC008148 (45836 until 46069) from Arabidopsis thaliana

Located on chromosome 1, on BAG clone F27J15 accession AC016041 (90633 until 90932) from Arabidopsis thaliana

Located on chromosome 1, on BAC clone F9N12 accession AC022355 (48056 until 48298) from Arabidopsis thaliana

Located on chromosome 1, on BAC clone F2P9 accession AC016662(57033 until 57356) from Arabidopsis thaliana

Additionally, a number of expressed sequence tags (EST's) and genes with previously unknown functions that were found in the database, belong to the LLP family, based on the criteria mentioned above. These include:

thaliana, Columbia Col-0, rosette-2 Arabidopsis thaliana cDNA clone 701546165, mRNA sequence gi|58454631|gb|AI998558.1|AI998558[5845463]

thaliana, Ohio State clone set Arabidopsis thaliana cDNA clone 701496429, mRNA sequence gi|5840376|gb|AI993471.1|AI993471[5840376]

Zea mays endosperm cDNA library from Schmidt lab cDNA, mRNA sequence gi|4887284|gb|AI677383.1|AI677383[4887284]

Z.mays mRNA for ESRa1 protein gi|2340960|emb|X98495.1|ZMRESRA1[2340960]

Z.mays mRNA for ESR2c1 protein |gi|2340958|emb|X98498.1|ZMRESR2C1[2340958]

Z.mays mRNA for ESR1c1 protein gi|2340956|emb|X98496.1|ZMRESR1C1[2340956]

Z.mays ESR3g2 gene, done L42a4 gi|2340954|emb|X99970.1|ZMESR3G2[2340954]

Z.mays ESR2g2 gene, clone L42a14 gi|2340952|emb|X99969.1|ZMESR2G2[2340952]

Z.mays ESR1g2 gene, clone LA2a6 gi|2340950|emb|X99968.1|ZMESR1G2[2340950]

Z.mays ESR2g1 gene gi|2340948|emb|X98499.1|ZMDESR2G1[2340948]

Z.mays ESR1g1 gene gi|2340946|emb|X98497.1|ZMDESR1G1[2340946]

Rice cDNA from immature leaf including apical meristem Oryza sativa cDNA clone E51222_{—}2Z, mRNA sequence gi|3763791|dbj|AU030543.1|AU030543[3763791]

Cotton Six-day Cotton fiber Gossypium hirsutum cDNA 5′, mRNA sequence

gi|6462118|gb|AW187682.1|AW187682[6462118]

Soybean Glycine max cDNA clone GENOME SYSTEMS CLONE ID: Gm-c1016-2901 5′, mRNA sequence gi|6094825|gb|AW119439.1|AW119439[6094825]

The criteria used to identify these LLP proteins can be used to recognise new members of the LLP family as they appear in public databases.

### EXAMPLE 6

### Isolation of Differentially Expressed Genes from the B. napus Microspore Embryogenesis System

As said, microspores of B. napus isolated at the stage around the first pollen mitosis were cultured in vitro at either 32° C. or at 18° C. (Custers et al., 1994). The higher temperature leads to a high frequency of embryo formation (sporophytic development) and the lower temperature leads to pollen maturation (gametophytic development, FIG. 1). Samples were collected at various time points (8 hr, 10 and 16 days) after initiation of the culture and analyzed for changes in gene expression using DD-PCR analysis. These time points were selected as being the minimum embryo induction stage (8 hr), the pattern formation stage (transition from globular to the heart shape, 10 days) and the differentiation stage (torpedo embryos, 16 days). To avoid the occurrence of non-embryogenesis but heat-shock related genes, microspores treated at 41° C. for 45 min were used as an additional control. Under such condition no embryogenesis was observed. More than 100 bands that showed increased or decreased expression in embryogenic culture were excised from DD-PCR gels, amplified by PCR and used as probes on Northern and reverse Northern analysis. Amplified fragments showing an expression pattern consistent with the original DD-PCR expression pattern was selected for further analyses. Sequence information was obtained from 82 bands and used to query publicly available sequence databases. Here we present a further characterization of one of these isolated genes, LLP1.

### EXAMPLE 6

### Identification of LLP1, a Gene Encoding a Small Protein with Signal Peptide

The LLP1 cDNA fragments was expressed in microspore-derived embryos of B. napus 10 days after the 32° C. induction treatment (globular to heart-shaped stage), but not in freshly isolated microspores (T=0), nor in microspores cultured at 18° C. or 41° C. or in leaves (FIG. 2, indicated by an arrow). This 368 bp DD-PCR fragment was sequenced after re-amplification and cloning (FIG. 3, bottom strand). To obtain a full-length LLP1 cDNA, we screened a cDNA library prepared from globular to heart-shape microspore-derived embryos using the LLP1 DD-PCR fragment as a probe. A 417 bp cDNA FIG. 3, topstrand) with a single open reading frame (ORF) was identified. This cDNA encodes a predicted 8.3 kDa peptide with 74 amino acids (FIG. 3, underlined). Analysis of the LLP1 protein using SignalP program (http://www.cbs.dtu.dk/services/SignalP/) indicated that, with 99.6% probability, LLP1 has a 23-amino acid hydrophobic transit signal peptide at its N-terminus (FIG. 3A, sequence before the [symbol] . Signal peptides control the entry of proteins to the secretion pathway (Nielsen et al., 1997) and are cleaved off during the transfer from the cytoplasm to the outside of the cell. Cleavage of the LLP1 signal peptide would produce a mature protein of only 51 amino acids.

Queries using the LLP1 sequence to protein and expressed sequence tag (EST) databases revealed no significant similarity with known proteins or cDNAs. However, comparison of LLP1 with DNA sequences in the GenBank database revealed homology with a recently sequenced Arabidopsis P1 genomic clone (MUJ8) located on chromosome 3 (37 cM on physical map). This region of the genomic DNA in Arabidopsis has one ORF with three candidate start codons. Structural comparisons of the Arabidopsis ORF with the B. napus LLP1 gene suggests that the second start codon is functional in this sequence, resulting in a peptide with the same length as the LLP1 protein (FIG. 13, AtLLP1.PRO). We named the Arabidopsis orthologue as AtLLP1. Both LLP1 and AtLLP1 lack introns. Interestingly, no match to the AtLLP1 gene was found among 114,351 ESTs available in Arabidopsis database, although RT-PCR showed clearly the existence of the transcript (data not shown). LLP1 is readily detectable by Northern analysis (see below) and is therefore not likely to be under represented due to it's abundance. A more likely explanation for the under representation of LLP1 ESTs could be that most cDNA libraries are constructed using fractionated cDNA, therefore genes like AtLLP1 with short transcripts may present in these libraries in very low abundance. Furthermore, due to its small ORF, the AtLLP1 gene has not been annotated as encoding a gene by the Arabidopsis genome-sequencing project. This could be a common problem for small unknown proteins.

SignalP analysis showed that AtLLP1 has 99.8% probability of carrying a 24amino acid signal peptide at its N-terminal. Over the 225 bp coding region, these two peptides shared 76.4% and 68% sequence identity at the DNA and protein level respectively. Southern blotting (data not shown) and database searching in the complete Arabidopsis genome sequence showed that AtLLP1 is a single copy gene located at the 37 cM position on chromosome 3. The map position is consistent with our data obtained from the analysis of recombinant inbred lines, which was carried out before this part of the genome was sequenced (data not shown).

### EXAMPLE 8

### LLP1 Shares Sequence and Structural Similarity with CLV3 and ZmESR Proteins

Protein sequence alignment between LLP1 and AtLLP1 showed that the longest stretch of conserved amino acids was present at the C-terminus (FIG. 12). We then used a 31-amino acid C-terminal peptide sequence to query public databases and found 18 other similar genes in Arabidopsis genome. Additionally, we also found some matching ESTs from Arabidopsis, tomato, soybean, medicago and cotton and some genomic sequences with similar ORFs (FIGS. 12 and 13). Alignment of these proteins revealed a conserved motif, KRXXPXGPXPLH, was present in all four proteins (FIGS. 12 and 13). This motif has not been previously described. We termed it the LLP box. Among these related sequences two genes, CLV3 from Arabidopsis and the ZmESR from maize, have been studied before although no linkage between these two genes was observed earlier. The TRIP box provided here allows us to identify the new gene family. CLV3 is the first protein ligand identified from higher plants, and interacts with the CLV1/CLV2 receptor kinase complex to mediate signal transduction within shoot apical meristems (Fletcher et al,, 1999). The ZmESR protein is encoded by a gene expressed in a restricted region of endosperm around the embryo (Opsahl-Ferstad et al,, 1997). Outside of the LLP box, the LLP1 proteins showed weak similarity with CLV3 (FIG. 3B, indicated in bold), but not with ZMESR. In the LLP1 and the AtLLP1 proteins, LLP box is located two amino acids before the C-terminal whereas in ZmESR the LLP box is located 43-AA before its C-terminal end. CLV3 has an additional 16-amino acids after the LLP box (FIGS. 12, 13). As said in Fletcher et al, the CLV3 gene encodes a protein of 96 amino acids that was thought to show no appreciable similarity to other sequences or sequence motifs of known functional domains, consequently, gaining the insight of a group of proteins sharing a common feature, namely the LLP box, and a common action mechanism (binding to a receptor and eliciting a phenotypic response) is provided herein for the first time. The cloning of CLV3 thus allowed Fletcher a view of meristems as collections of intercommunicating cells, each sythesizing and secreting its own set of protein ligands and responding to its neighbors through action of its own complement of transmembrane receptor kinases, however, even though it is well-understood that other protein ligands must exist in many proteins (inside or outside the meristem, for that matter), Fletcher et al provide no method for finding or identification of such ligand. Similarly, in Opsahl-Ferstad et al. A number of maize genes were identified with a specific expression pattern, signal sequence and size. Conserved domains found among these genes do not include the LLP box, this is located in what they define as the variable region. functions of Esr as proposed by the author include physical separation of embryo and endosperm (a structural role in the cell wall), or nutrition of the embryo (to be taken up and consumed). No mention was made of a possible function in signal transduction as a ligand to direct differentiation of either embryo or endosperm.

### EXAMPLE 9

### LLP1 is Expressed in a Defined Small Number of Cells During Embryonic and Post-embryonic Development

The DD-PCR experiment showed the expression of LLP1 in microspore-derived embryos, but not in microspores/pollen and leaf tissue (FIG. 2). Northern blotting was used then to further characterize the LLP1 expression pattern in additional tissues. Northern blot analysis showed relatively high amounts of LLP1 mRNA in the globular to heart-shape staged embryos and in young flower buds (1-5 mm in size), lower levels in older flower buds (5-8 mm) containing binucletae to trinucleate pollen, and almost undetectable levels in open flowers at the anthesis stage (FIG. 4). Within flower buds, expression was detected in pistils, but not in anthers and petals (FIG. 4). No detectable signal was observed in leaves.

To study LLP1 expression in more detail, a 1,060 bp genomic sequence (GenBank accession number AF343658, from 0 to 1,060 bp) located up-stream of the LLP1 start codon was isolated from B. napus by genome walking, fused to the E. coli β-glucuronidase A (GUS) reporter gene and transformed to Arabidopsis.

We analyzed LLP1 expression during embryonic and post-embryonic development in several transgenic Arabidopsis lines. The result was consistent with the Northern blot analysis, and among different transgenic lines. To define the precise expression pattern of LLP1 during embryogenesis, zygotic embryos from transgenic plants were excised from seed and then stained for GUS activity. Hoyer clearing procedure caused the diffusion of the GUS staining, therefore the results are also presented diagrammatically based on observation under a dissection microscope. As shown in FIG. 5A, LLP1 expression was first detected in the upper region of the late globular embryos. At the heart-shape stage, the GUS staining was restricted to a few cells at the top and at the abaxial side of the cotyledon primordia (FIGS. 5A and B). Further development of the embryo led to a change of the LLP1 expression to a narrow tier of cells at the edge of the cotyledon in torpedo-shaped embryos (FIG. 5A). At the bent cotyledon stage LLP1 expression was localized to a ring-shaped region at the base of each cotyledon, but was absent from the shoot meristem itself (FIGS. 5A and D). During seed germination, LLP1 expression was observed in the aleurone, a single layer of endosperm located between the testa and the embryo (FIGS. 5, E and F).

Freshly germinated seedlings showed no GUS expression. The first detectable GUS signal was seen in the root hair region when the main root was longer than 1 cm (5 days after plating). A sharp difference in GUS staining was seen between the root, which was stained very strong, and the hypocotyl that was always negative for GUS activity (FIG. 6B). Within the root, LLP1 expression was excluded from the epidermis layer and, no GUS staining was seen in the root hairs (FIGS. 6, B and C). Occasionally, LLP1 expression was observed in the quiescent center of the primary root (FIG. 6D). We are not sure if it is from the promoter activity or just background staining sinc 1) it is not consistent; 2) other researchers have observed such background activity before. Along the long axis of the root, LLP1 expression was seen only in the well-developed root hair region along a total length of 1 cm or less (FIG. 7). Neither the root tip, nor the elongation zone and the secondary thickening zones exhibited any GUS staining (FIGS. 6, E and A). Although LLP1 expression was observed in all cell layers in freshly germinated primary roots except epidermis (FIG. 7D), at later stages the expression was restricted to the pericycle layer outside the xylem elements (FIGS. 7, B and C). In radial sections, LLp1 expression was observed in two or three pericycle cells facing the protoxylem, whereas the pericycle cells next to the protophloem were always negative (data not shown). In Arabidopsis, the central cylinder is of the diarch type i.e. with two protoxylem elements and at a right angle to 2 protophloem elements. The pericycle at the outmost layer of the central cylinder is composed of an average of 12 cells in circumference and the lateral root always initiates from the pericycle cells that face to the protoxylem (Dolan et al., 1993). During lateral root formation, we observed that LLP1 expression was completely down-regulated in the region, as well as in the cells adjacent to the protoxylem (FIG. 6C). The LLP1 expression pattern, together with the different potential in lateral root induction in this layer, indicates that different cells in the pericycle ring may have different developmental potentials in relation to their positions. The expression in lateral roots re-assumed as they matured enough and became covered with root hairs. In summary, LLP1 expression in roots is associated with few pericycle cells in the maturation zone. This region of the root is normally covered with root hairs and functions predominantly for nutrient intake from the soil.

In above ground tissues LLP1 expression was restricted to floral and inflorescence meristems. The first detectable GUS signal was seen in the axillary inflorescence (also called paraclade) primordia of 8-day old seedlings carrying 3-4 leaves. The primary vegetative meristem did not show any expression before switching to an inflorescence meristem. The determination of the inflorescence meristem may occur earlier in the axillary buds than in the primary vegetative meristems, since all the axillary buds at the time of initiation are determined to form a paraclade (inflorescence shoot). In Arabidopsis (C24), each axillary bud will give rise to one paraclade with 3-5 cauline leaves before the production of an indefinite number of flowers. Once the flower starts to form, no additional cauline leaves will be produced. In the young axillary buds, LLP1 expression was observed in the periphery of the meristem, at the point where the cauline leaves will emerge (FIG. 8) and appears to be restricted to the L1 layer. This expression pattern continued until the young leaf primordia were formed and was switched off before the expansion of the leaves (data not shown). The central inflorescence meristems were always negative in LLP1 expression (data not shown).

In floral meristems, LLP1 expression was first observed in stage 2 flower (Smyth et al., 1990) buds at the regions where sepal primordia will be formed (data not shown). This expression pattern continued until stage 3, which marks the sepal primordia, at which point we observed asymmetrical LLP1 expression between the medial and. radial sepals. LLP1 expression appears to initiate earlier and is stronger in the radial sepal, which also emerges before the medial sepal. Such an asymmetrical flower development has not been observed in the morphological analysis carried out in Arabidopsis by Smyth et al (1990), but was previously demonstrated in B. napus (Polowich and Sawhney, 1986). In stage 4 flower buds, LLP1 expression was restricted to the grooves between the sepal primordia and the central meristem and disappeared completely in stage 6 flower buds when the petal and stamen start to form (data not shown). In stage 7 to 11 floral buds, LLP1 expression was only observed at the top of the pistil where the stigma will form. The expression of LLP1 in flower buds is switched off shortly before the flower opens.

### EXAMPLE 10

### Identification and Characterisation of other LLPs in Arabidopsis Genome

FIG. 14 shows the criteria we used to search various databases; FIG. 12 shows LLP proteins identified in arabidopsis genome. From the fully sequenced Arabidopsis genome, it is possible to see how many LLP genes are present. This has led to the identification of 19 LLPs. The map position of the LLP genes were showed in FIG. 32. Although a few of the LLP genes have EST sequences available, none of these 19 LLP genes have been annotated as a gene by the genome sequencing groups. The distribution of these LLP genes seems not random. At the bottom of chromosome 1, there is a big cluster of LLP genes, no LLP has been found in chromosome 4 (FIG. 33). It is interesting to notice that except the functional conservation through this group genes, encoding small proteins with N-terminal signal peptide and C-terminal conserved LLP box, none of them have redundant copies (paralogues) in Arabidopsis genome. In another words, none of these LLPs shares more than 50% identity at the peptide level. This could be the nature for this group of genes. Several reasons can be proposed: 1) the secondary structure of these peptides is more important for their functions than the amino acid order. This has been seen in SMC proteins which have two rod regions have highly conserved coiled coil structure but flexible in primary sequence. 2) the sequence flexibility allows precise interaction with corresponding receptor kinases. 3) the critical importance of such proteins requires single copy in a plant genome.

### EXAMPLE 11

### Identification of LLP genes in other Plant Species

A similar search has been carried out in other available databases using the criteria we set up as mentioned above. This has led us to identify LLP genes in species such as rice, Medicago, tomato, etc. (FIG. 13). All these gene identified showed s structural conservation as those ones from Arabidopsis.

### EXAMPLE 12

### Ectopic Expression of LLP1 in Arabidopsis Leads to a Consumption of the Meristem Without Affecting the Induction of Lateral Roots and Side Shoots

A double enhanced 35S promoter, which is constitutively expressed in most plant tissue, was used to drive the expression of the B. napus LLP1 cDNA (35S::LLP1) in Arabidopsis. Among twenty-five independent transformants obtained, four lines (A, B, C and D) showed similar aberrant phenotypes: slow growth and late flowering. Bolting occurred only 40-45 days after seeds were planted, instead of 20 days in the wildtype. One line (Line D) was male and female sterile and gave no seed for further analysis in the next generation. Genetic analysis of the remaining three lines indicated that their phenotypes were inherited in a Mendelian fashion. Plating of single insertion heterozygous lines on kanamycin selection media showed that the phenotype is always associated with the transgene, the ratio is consistent with phenotype segregation in soil without kanamycin.

Dramatic changes in root development were observed in all four over-expression lines. Freshly germinated 35S::LLP1 seedlings showed little difference from the wildtype seedlings. However, root growth in 35S::LLP1 plants was retarded (FIGS. 23, A to D, 12-day old). Root hairs formation and the initiation of lateral roots were normal in 35S::LLP1 roots (FIGS. 23, B and D), further growth of the lateral stopped shortly after root hair formation. Consequently, 35S::LLP1 over-expression led to the formation of seedlings with short roots Fifteen days after seed germination, the transgenic plants had produced 4 to 8 short roots, with an average length of less than 1 cm, while in the same period of time the main roots in wildtype seedlings reached a length of more than 10 cm, with several side roots of different lengths. Root hairs in the over-expression lines were formed almost to the tip of the roots (FIG. 23D). 35S::LLP1 root tips also appeared to be narrow and pointed, as compared to the wildtype roots. Root geotropism was not affected in 35S::LLP1 seedlings (data not shown).

Tissue Clearing, followed by Nomarski microscopy of 35S::LLP1 roots showed that root meristematic tissue was gradually consumed during root growth and development and used to form differentiated cells. As shown in FIGS. 11E and F (7 days after plating), clear differences in the root region could be observed between wildtype and over-expression seedlings. In the wildtype roots, cells were arranged regularly with clear size and shape differences between the root cap, root meristem, elongation zone and maturation zone (FIG. 23E). In LLP1 over-expression plants the root meristem zone and the elongation zone became shorter, which was followed immediately by the formation of highly vacuolated cells which were typically seen in the root hair region (FIG. 23F). At this developmental stage the quiescent center was still recognizable. Ten days after germination treatment, the root meristem had almost entirely disappeared (FIG. 23G). Only a small number of meristematic cells were present in the root tip. These cells were adjacent to highly vacuolated cells that are normally located at the root hair region. The elongation zone and the quiescent center were hardly recognizable (FIG. 23G). The root meristematic cells and the quiescent center had totally disappeared in 2-week old 35S::LLP1 seedlings (FIG. 23H). All the cells in this region became highly vacuolated and exhibited a thickening of their cell walls. Xylem elements reached the central cell region (FIG. 23H, indicated by an arrowhead). Abnormalities were also observed in the root cap, although starch grains were still visible (data not shown). No evident difference in root structure was seen in wildtype root during this growth period (data not shown). The same meristem defect observed in primary roots was also observed in the secondary roots. In conclusion, ectopic expression of LLP1 under control of the 35S promoter appeared to have no influence on root induction, but it had a strong effect in promoting the differentiation of meristematic cells--meristem is consumed faster than it can be regenerated.

Ectopic expression of LLP1 led to similar changes in shoot and floral development observed for root development. All four independent transformants showed a short branch phenotype. Line A had a weaker phenotype than line B and C, and produced about 1-3 paraclades in total with relative high amount of seeds produced. These inflorescences stopped to form new flowers after producing 10 or less siliques each, instead of 30 to 40 each in the wildtype. Line B and C were almost completely male sterile, resulting occasionally in a small number of seeds (less than 30 per plant) under normal growth condition. These seeds probably originated from cross-pollination, since no viable pollen could be detected in the anthers of line B and C flowers. Genetic analysis was difficult to carry out with such a limited number of seeds. About one third of the flowers formed in plants from line B and C plants had pin-shaped pistils that showed no ovule development and consequently no seed formation (FIG. 10). The other two thirds of the flowers had normal pistils that were able to produce seeds if pollinated with wildtype pollen. No pin-shaped pistils were observed in line A, which had a weaker phenotype. Northern blot analysis revealed that the level of LLP1 mRNA in all these four lines was much higher than those transgenic lines with a wild-type phenotype (data not shown). The short branch and pin-shaped pistil phenotypes could be the consequence of the consumption of inflorescence and floral meristems, similar to what was observed in root meristems with the LLP1 over-expression.

Additionally, ectopic LLP1 expression of seems to stimulate the formation of paraclade from the axillary buds. Instead of the single paraclade normally produced from each wildtype axillary bud (FIG. 9), multiple paraclades were commonly formed in the 35S::LLP1 over-expression lines (B and C), particularly in the axils of cauline leaves (FIGS. 9B and C). Up to 7 paraclades were sometimes observed to regenerate from one axil (data not shown). These paraclades normally emerged sequentially, rather than simultaneously. The terminal flower 1 mutant also shows an increase in branch formation, however in this mutant, the multiple shoots are formed in the axils of rosette leaves, and only occasionally from cauline axils (Grbic and Bleecker, 2000).

### EXAMPLE 813

### Ectopic Expression of LLP1 Leads to Defects in the Formation of Continuous Vascular Network in Flowers

Aberrant vascular development was also observed in flowers of 85S::LLP1 over-expression lines. In the wildtype flowers, vascular bundles are formed at stage 9 by extension from the main stem up to flower buds (FIG. 14A). Xylem elements were established first in sepals and followed by pistils, stamens and petals, resulting in a complete vascular network. In 35S::LLP1 flower buds (lines B and C), regional vascular formation without connection to the stems was observed (FIGS. 11B and C). The failure to form a vascular connection seems to be associated with the formation of pin-shaped pistils, since this phenomenon was not observed in flowers with normal pistils. However, not all flowers with pin-shaped pistils have discontinuous vascular towards the main stem. Some flowers did form continuous xylem connections, although the number of xylem elements was reduced as compared to the wildtype. Local xylem formation as vascular islands was also observed in both sepals and petals (data not shown). Such vascular islands were observed in flowers with normal and pin-shaped pistils. No vascular bundle was formed within these pin-shaped pistils.

### EXAMPLE 14

### Expression of LLP2 Gene (Sense Strand) Under the control of Double Enhanced CaMV 35S Promoter

LLP2 coding region was amplified by PCR and cloned in both sense and anti-sense orientations to be expressed under the control of double enhanced CaMV 35S promoter (using the same over-expression vector mentioned above). Transgenic plants were obtained by selection on kanamycin-containing media. One over-expression plant showed defective in reproductive development (FIG. 24A). The plant continues produce leaves. Occasionally one of two flower can be formed. (FIG. 24B). Detailed observation showed that such flower has normal sepal and petal, but reduced number of stamen and no pistil (FIG. 24C). The inflorescence meristem terminated quickly before further flower formation (FIG. 24D).

### EXAMPLE 15

### Expression of LLP2 Anti-sense Under the Control of Double Enhanced CaMV 35S Promoter

Over-expression of LLP2 anti-sense under the control of double enhanced 35S promoter leads to plants with soft and short stems. Each inflorescence produces 2-6 siliques instead of 25 to 35 in the wildtypes. The number of seeds in each silique was also greatly reduced. It is likely that the over-expression of LLP2 anti-sense affected the vascular structure of the plants. Genetic analysis showed that the phenotype is associated with the T-DNA insertion. Tissue specific promoter could be used in combination of the LLP2 anti-sense gene to modify the vascular structure of other plant species.

### EXAMPLE 16

### Expression of LLP11 Gene Under the Control of Double Enhanced CaMV 35S Promoter

LLP1 coding region was amplified by PCR from genomic DNA. The gene was expressed under the control of double enhanced CaMV 35S promoter (using the same over-expression vector mentioned above). 78% T0 plants over-expressing LLP11 gene (sense strand) showed phenotypes. Based on the phenotype differences, the T0 plants can be divided in three classes: light, medium and severe phenotype lines (FIG. 25). The “light phenotype” plants can produced a few inflorescence although the primary one often stopped prematurely. The “medium phenotype” plants showed greatly reduction on inflorescence formation. Normally a very or a few very short inflorescence can be produced, with few siliques. The “severe phenotype” plants do not form any inflorescence, therefore, can not be carried to the next generation. Several “light phenotype” plants were analyzed in the following generations since enough seeds were available. Among the lines analyzed, we observed two different phenotypes that are slightly different from the phenotypes we observed in the To generation: low fertility (FIG. 26A) and slow growing and reduced inflorescence formation phenotype (FIG. 26B). In some lines both phenotypes can be observed and in other lines only one phenotype was observed. Genetic analysis showed clearly that the low fertility and slow growing phenotype were caused by over-expression of the LLP11 gene, since both traits showed to be dominant and linked to the T-DNA in segregation. The slow growing phenotype can be seen in both root and shoot development, producing plants with short roots and small leaves. Some low fertility lines (#67-6, FIG. 26A) showed no reduction on vegetative growth. The plants have long paraclade with very short siliques (because of no or a few seeds produced in each silique). It is possible that the LLP11 genes (sense and anti-sense approaches) can be used in combination with different promoters to control growth behavior and pollen development.

### EXAMPLE 127

### Analysis of the Expression Pattern of LLP12 Using GUS Fusion Construct

The promoter region of LLP12 (1 kb before ATG) was cloned in front of the GUS reporter gene in a pBINPLUS vector. Transgenic plants were obtained using the flower dip method mentioned above. GUS expression analysis was carried out in leaves, stems, axillary buds, flowers and siliques in 30 independent transgenic lines. The results showed, with certain variation in GUS staining, that the LLP12 was expressed in immature pollen grains and the pedicel region (the connection between flower and the stem) of the flowers (Indicated by diagrammatic drawing in FIG. 27). GUS analysis in root development will be carried out in the near future.

### EXAMPLE 18

### Expression of LLP12 Gene Under the Control of Double Enhanced CaMV 35S Promoter

Several transgenic plants expressing LLP12 gene showed more or less the same phenotype. The primary shoots were stopped early and multiple side shoots were formed afterward (FIGS. 28, A and B). The plants have very thin and short inflorescences, with no (FIG. 28B) or a few seeds (FIG. 28A) produced. The reduced seedset seemed to be caused by male sterility since seeds can be produced when cross-pollinated with WT pollen. Flower development was normal. Phenotype segregation can be seen clearly in the in the next generation when seeds were planted on germination plates with or without the selection agent (Km). During the seedling stage, the transgenic plants have smaller rosette leaves and reduced root elongation. The phenotype segregation could also been seen clearly when seeds were sowed directly in soil (FIG. 29C). At later stage of the inflorescence development, the paraclade showed zigzag arrangement (FIGS. 30, A and B). Instead of new flowers formed from the side of the inflorescence, in this case, the new flowers formed at the terminal position of the paraclade, whereas the inflorescence were produced at the side. The pedicel (the joint between stem and flower or silique) was also much shorter (FIG. 30B) than that in the WT plant. The low fertility and short peduncle phenotype seem consistent with the expression pattern of the LLP12 gene. The retarded growth of pedicel may be associated with the suppression function generally seen in most LLP genes. Genetic analysis showed that such phenotypes are dominant traits and linked to the T-DNA (FIG. 31, WT plants have been removed from the top picture). The male sterility caused by LLP12 over-expression could be used to modify the reproduction behavior or in hybrid seed production.

### EXAMPLE 19

### RT-PCR to Test if the LLP ORFs are Real Genes, and Where do they Expressed

Since most of the LLP genes were identified from the genome sequence based on the criteria we set up, it is not sure if all of them are real expressed genes. RT-PCR was used to check the expression profile of these ORFs. Total RNAs were isolated from various tissues of Arabidopsis and treated with DNase to remove contamination from genomic DNAs. RT-PCR was performed using poly(T) as a primer. For the positive control and the quantitative measurement, ACTIN8 gene was used as positive control since it is a ubiquitously expressed gene. Two primers, one located at the beginning of the ORF and one before the stop codon, were used to perform the PCR reaction. When RNA was used to do the PCR, no product has been seen, indicating that genomic contamination have been removed. Positive control was carried out using genomic DNA These experiments revealed for example that LLP2, LLP9, LLP12 and LLP18 are genes with different expression profiles. LLP2 was expressed in all tissues tested. LLP9 was only expressed in different stages of flowers, not in roots, leaves, stems, etc. LLP12 showed higher expression in different stages of flower, but also in other tissues tested. LLP18 showed expression only in roots. Two genes, LLP5 and LLP7, showed negative in the RT-PCR analysis in the tissues tested. In summary, These RT-PCR experiments showed that most LLP genes identified using the criteria we established are genes of which the expression is different from one another.

### Experimental Procedures

Plant Material and Microspore Culture

The cultivation of the doubled haploid Brassica napus L. cv. Topas plants and the isolation of microspore and pollen grains was performed as described. Plants were raised all year round in a phytotron room at 18° C. with a 16 h photoperiod. Microspores and pollen were isolated by disrupting flower buds with a pestle in NLN medium (Lichter, 1982) containing 13% (w/v) sucrose (NLN13). Late unicellular microspore and early bicellular pollen were cultured in NLN13 medium at a density of 40,000 cells/ml, either at 18° C. (gametophytic development) or at 32° C. (embryogenic development).

Nucleic Acid Isolation

Total RNA from microspore cultured at 18° C. (8 h), 32° C. (8 h) or 41° C. (45 min) was isolated using an extraction buffer containing a 1:1 mixture of phenol and 0.1 M LiCl, 10 mM EDTA, 1% SDS, 0.1 M Tris-HCl (pH 8.0). One ml of hot (60° C.) extraction buffer was added to the microspore pellet (approx. 10⁶ microspore) and the homogenate was rigorously vortexed in the presence of glass beads. After centrifugation the aqueous phase was extracted with an equal volume of chloroform and the RNA was precipitated at −20° C. by the addition of ⅓ vol of 8 M LiCl. The pellet was washed with 70% ethanol dried and dissolved in diethylpyrocarbonate (DEPC)-treated water. All other total RNA samples were obtained by grinding the plant material in liquid nitrogen with a mortar and pestle, and subsequent extraction of the fine powder using TRIZOL reagent (Gibco-BRL). Genomic DNA was isolated from leaf tissue according to Fulton et al., 1995, and digested with the specified restriction enzymes according to procedures suggested by the manufacturer (Gibco-BRL).

Differential Display

Differential display (Liang and Pardee, 1992) of mRNA was performed using RNAmap Kit B (GenHunter, USA) according to the manufacturer's recommendation. Total RNA from freshly isolated microspore, microspore cultured at 18° C. (8 h), 32° C. (8 h, 10 d, 16 d) or 41° C. (45 min), and leaf tissue of B. napus was isolated as described above and DNAse I treated using the MessageClean Kit (GenHunter). Differential display was carried out on two independent 8 h cultures of 18° C. and 32° C. A real heat-shock was given by treatment of microspore at 41° C., a condition that does not lead to embryogenesis in microspore of this developmental stage. DNAse-free total RNA samples (0.2 μg) were used for the first strand cDNA synthesis. Four T₁₂MN anchor primers (where M is degenerate A, C, G and N is either A, C, G or T) were used in four reverse transcription (RT) reactions. PCR amplification of one-tenth of the first-strand synthesis cDNA products was done in the presence of [α-³³P]dATP. Five decamers (AP₆ to AP₁₀) were used in combination with the respective T₁₂MN. All PCR steps were performed using the Perkin-Elmer GenAmp 9600 system and AmpliTaq polymerase from Perkin-Elmer. The amplified [α-³³P]dATP labeled cDNAs were resolved on 6% denaturating polyacrylamide gels containing 7 M urea. After drying the gels on Whatman 3MM paper and autoradiographic detection of bands, differentially expressed cDNAs were excised and eluted according to the manufacturer's instructions. cDNAs were then re-amplified using the same PCR conditions and primers as before. PCR products were analysed on a 1.2% agarose gel and cDNA fragments of interest were eluted and cloned into the pGEM-T vector (Promega). To confirm the differential display pattern the cloned cDNAs were used as probes for RNA blot hybridizations.

DNA and RNA Gel Blot Analyses DNA fragments were separated in 1% agarose and transferred overnight onto Hybond-N⁺ (Amersham) by capillary blotting with 20×SSC. For RNA gel blot analysis, 10 μg of total RNA was denatured with glyoxal prior to electrophoresis and blotting onto Hybond-N⁺ membrane. After ultraviolet cross-linking the membranes were hybridized with a [³²P] random-primer-labeled probe of the DD-clone of BnLLP1. Membranes were hybridized overnight at 65° C. in 10% dextran sulphate, 1% SDS, 1 M NaCl, 50 mM Tris-HCl (pH 7.5) and washed first 30 min twice at moderate stringency (65° C., 2×SSC, 1%SDS), followed by two 30 min high-stringency washes (65° C., 0.2×SSC, 0.5%SDS).

cDNA Library Construction and Screening

Poly(A)⁺ RNA was isolated from total RNA of globular to heart stage B. napus microspore embryos using Poly(A) Quik columns (Stratagene). Five μg poly(A)⁺ RNA was used as starting material for the construction of an Uni-ZAP XR cDNA library (Stratagene). Approximately 10⁶ plaques were screened under high-stringency conditions with the cDNA as isolated by DDRT-PCR (FIG. 3 ). One positive clone was isolated, purified and sequenced (FIG. 3).

Isolation of Promoter Sequence

The Universal Genome Walker Kit (Clonetech) was used to isolate genomic DNA fragments lying upstream of the BnLLP1 ATG start codon. Pools of uncloned, adaptor-ligated Brassica napus cv Topas genomic DNA fragments were constructed and used to isolate BnLLP1 genomic sequences by nested PCR. The primary PCR made use of the outer adapter primer (AP1) supplied by the manufacturer and a BnLLP1 specific primer with the sequence:

5′-CCATTCTTCATCAGCAAACTCCGAAATGA-3′

The nested PCR made use of the nested adapter primer (AP2) supplied by the manufacturer and a BnLLP1 specific primer with the sequence:

5′-CAGAAAAGAGGAAGCCAATATCAAACTC-3′

The primary PCR mixture was then diluted 1:50 and used as template for nested PCR. Both the primary and nested PCRs were performed as recommended by the manufacturer. The nested PCR products were cloned into the pGEMT vector (Promega) and sequenced. PCR products corresponding to the 5′ untranslated genomic region of BnLLP1 cDNA were identified (FIG. 16).

Plasmid Construction for Plant Transformation

The construction of a plasmid vector containing the BnLLP1 cDNA under control of the double cauliflower mosaic virus 35S promoter with d AMV translational enhancer was as follows. The complete coding region of BnLLP1 was already cloned into the GST fusion vector pGEX4T-2 (Amersham Pharmacia Biotech). This plasmid was cut with the restriction enzymes BamHI and XhoI. The 231 bp BnLLP1 fragment was isolated and ligated into the vector pGD121 (containing a double 35S promotor with AMV enhancer and a pBINplus backbone), already cut with the restriction enzymes BamHI and XhoI. This construct was confirmed by sequencing, and transformed to A.tumefaciëns C58PMP90.

Promoter-GUS Construct

The promotor BnLLP1-GUS was made as follows; a 1060 bp BnLLP1 promotor fragment (obtained by genome walking) cloned in pGEM-T (PROMEGA) was used for this construction This construct was used in a PCR with the primers: P312-1 5′-CGCTCTAGAGTTCTATCTTTGTCAAAAAAAA-3′ anneals on the promotor, just before the ATG.

P312-2 5′-ATATAAGCTTACTATAGGGCACGCGT-3′ anneals on the genome walker adaptor.

As proofreading polymerase Pfu (STRATAGENE) was used. PCR protocol: 45 seconds at 94° C., 60 seconds at 40° C., 4.5 minutes at 72° C. [cycle repeated twice] followed by a 45 seconds at 94° C., 60 seconds at 54° C., 4.5 minutes at 72° C. [cycles repeated 18 times] followed by 3 minutes at 72° C. and a 4° C. hold. The obtained fragment was cut with the restriction enzymes HindIII and XbaI, and ligated into the pRAP2T/GUS vector (containing GUS intron and the NOS terminator and a pUC vector as backbone) that was already digested with HinDIII and XbaI. This constuct was digested with PacI and AscI and a fragment containing the BnLLP1 promotor, GUS-intron and the NOS terminator was isolated and ligated into pBINplus, digested with PacI and AscI. The obtained vector was confirmed by sequencing and transformed to A.tumefaciëns C58PMP90.

Plant Transformation

Arabidopsis thaliana ecotype C24 was used as the recipient in transformation experiments. Plants were transformed using the flower dip method described in Clough and Bent (1998).

Cryo-electron Microscopy

Plant materials were glued to a copper stub using conductive carbon glue and immediately frozen in liquid nitrogen. The sample was then transferred to a low temperature field emission scanning electron microscope (LT-FESEM, JEOL JSM 6300F) equipped with an Oxford cryochamber. After a light coating with argon gas the samples were observed and pictures were taken with a digital camera.

TABLE 1
Examples of genes regulating plant development

| Gene function | Name | Protein identity | References |
|---|---|---|---|
| Cotyledon | LEC1 | CCAAT box transcription | Lotan et al, 1998 |
| identity | | factor | |
| | CUC2 | putative transcription factor | Aida et al, 1997 |
| Floral organ | AG | MADS transcription factor | Yanofsky et al, 1990 |
| identity | | | Mandel et al, 1992 |
| | AP1 | transcription factor | Jufuku et al, 1994 |
| | AP2 | transcription factor | Weigel et al, 1992 |
| | LFY | transcription factor | |
| Shoot meristem | STM | KNOTTED-like transcription | Long et al, 1996 |
| identity | factor | | |
| | CLV1 | receptor kinase | Clark, et al, 1997 |
| | CLV2 | receptor kinase | Jeong et al, 1999 |
| | CLV3 | ligand protein | Fletcher and |
| | | | Meyerowitz, 1999 |
| Abaxial-adaxial cell fate | FIL | putative YABBY transcription | Sawa et al, 1999 |
| | | factor | |
| Vascular | MP | auxin-responsive | Hardtke and |
| development | | transcription factor | Berleth, 1998 |
| Signal | BRI | receptor kinase | Li and Chory, 1997 |
| transduction | | | |

### References

Aida, M., Ishida, T., Fukaki, H., Fujisawa, H and Tasaka, M. (1997) Genes involved in organ separation in Arabidopsis: an analysis of the cup-shaped cotyledon mutant, Plant Cell 9:841-857.

Chang, C. and Meyerowitz, E M (1995) The ethylene hormone response in Arabidopsis—an eurokaryotic 2-component signalling system. Proc. Natl. Acad. Sci USA 92: 4129-4133.

Citovsky, V and Zambryski, P. (1991) How do plant virus nucleic acids move through intercellular connections. BioAssays 13: 373-379.

Clark, S. E., Williams, R. W., Meyerowitz, E M. (1997) The CLAVATA1 gene encodes a putative receptor kinase that controls shoot and floral meristem size in Arabidopsis. Cell 89:575-585.

Clough, S. J. and Bent, A. F. (1998) Floral dip: a simplified method for Agrobacterium-mediated transformation of Arabidopsis thaliana. Plant J. 16: 735-743.

Fletcher, J. C., Brand, U., Running, M. P., Simon, R. and Meyerowitz, E. M. (1999) Signaling of cell fate decision by CLAVATA3 in Arabidopsis shoot meristems. Science 283:1911-1914.

Jinn, T.-L., Stone, J. M. and Walker, J. C. (2000) HAESA, an Arabidopsis leucine-rich repeat receptor kinase, controls floral organ abscission. Genes and Development 14:108-117.

Kakimoto, T. (1996) Cki1, a histidine kinase homolog implicated in cytokinin signal-transduction. Science, 274:982-985.

Ku, T., Mitsukawa, N., Oosuin T., Matsuura, R., Whittier, R. F. and Komeda, Y. (1996), The Arabidopsis ERECTA gene encodes a putative receptor protein kinase with extracellular leucine-rich repeats. Plant Cell 8:735-746.

Liang, P., and Pardee, A. B. (1992) Differential display of eukaryotic messenger RNA by means of the polymerase chain reaction. Science 257: 967-971.

Lichter (1982). Induction of haploid plants from isolated pollen of Brassicia napus. Z.Pflanzen-Physiol. 105: 427-434.

Lawton, M A, Yamamoto, R T, Hanks, S K, Lamb, C J (1989) Molecular cloning of plant transcripts encoding protein kinase homologs. Proc. Natl. Acad. Sci. USA 86:3140-3144.

Lucas W. J., Bouche-Pillon, S., Jackson, D. P., Nguyen, L., Baker, L., Ding, B. and Hake, S. (1995) Selective trafficking of Knotted1 homeodomain protein and its mRNA through plasmodesmata. Science 270:1980-1983.

Hake S. and Char, B. (1997) Cell-cell interactions during plant development. Genes and Development 11:1087-1097.

Sawa, S., Watanabe, K, Goto, K, Kanaya, E., Morita, E. H., Okada, K (1999) FILAMENTOUS FLOWER, a meristem and organ identity gene of Arabidopsis encodes a protein with a zinc finger and HMG-related domains. Genes Dev., 13:1079-1088.

Van den Berg, C. V., Willemsen, V., Hage, W., Weisbeek, P. and Scheres B. (1995) Cell fate in the Arabidopsis root meristem determined by directional signalling. Nature 378:62-65.

Fletcher, J. C. and Meyerowitz, E. M. (2000) Cell signalling within the shoot meristem. Current Opinion in Plant Biology, 3:23-30.

Fulton, T. M., Chunwongse and Tanksley (1995) Microprep protocol for extraction of DNA from tomato and other herbaceous plants. Plant Molecular Biology Reporter, 13(3): 207-209.

Li, J. M. and Chory, J. (1997) A putative leucine-rich repeat receptor kinase involved in brassinosteroid signal transduction. Cell, 90: 929-938.

### FIGURE LEGENDS

FIG. 1. A diagram showing the microspore embryogenesis system we used to identify genes involved in embryogenesis. Late uni-cellular microspores and early bi-cellular pollen isolated from B. napus ‘Topas’ developed into embryos when cultured at 32° C., while the same population of cells continued gamethophytic developement into mature pollen when cultured at 18° C. Embryo or pollen materials can be harvested at different stages from these two conditions for RNA isolation.

FIG. 2. Identification of the LLP1 clone using differential display technique. A portion of a differential display gel showing the presence of LLP1 cDNA in 10-day and 16-day microspore-derived embryos. The RNA samples were prepared from the following materials:

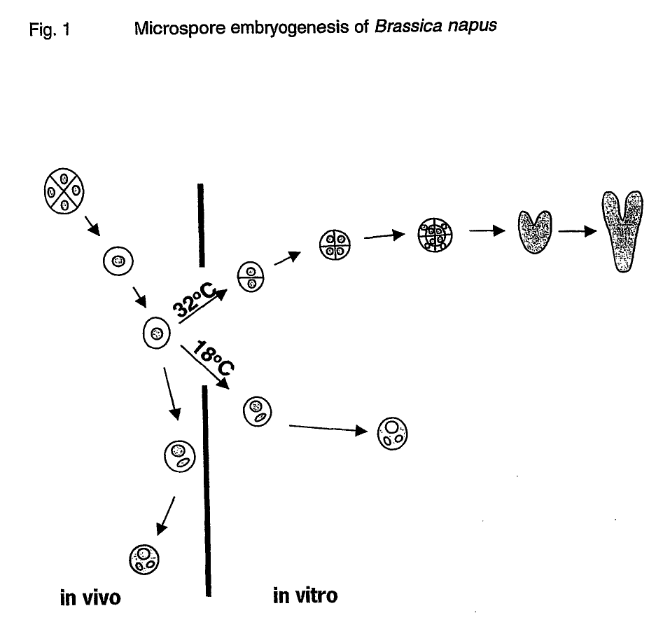

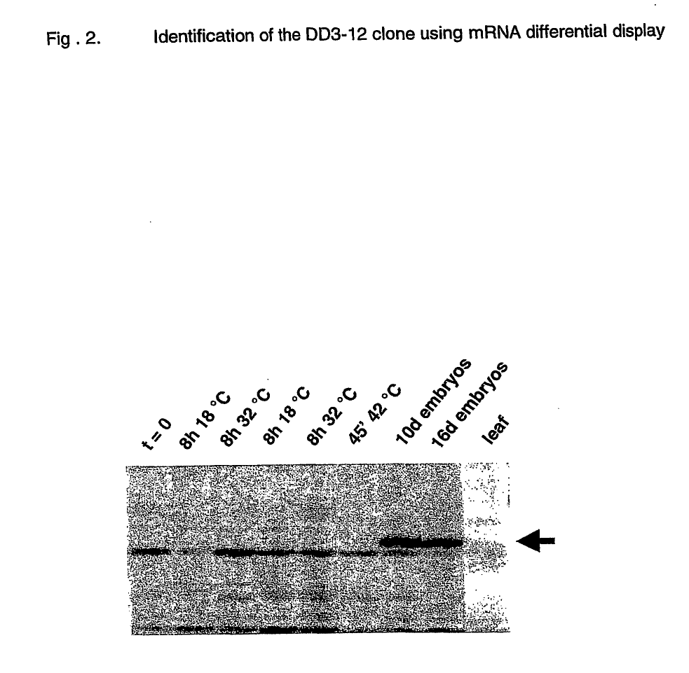

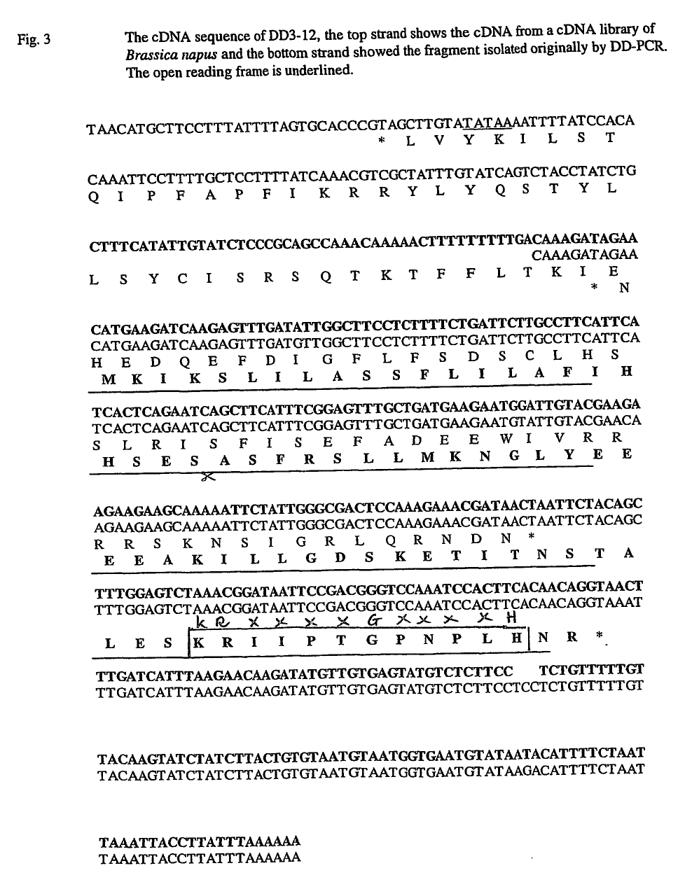

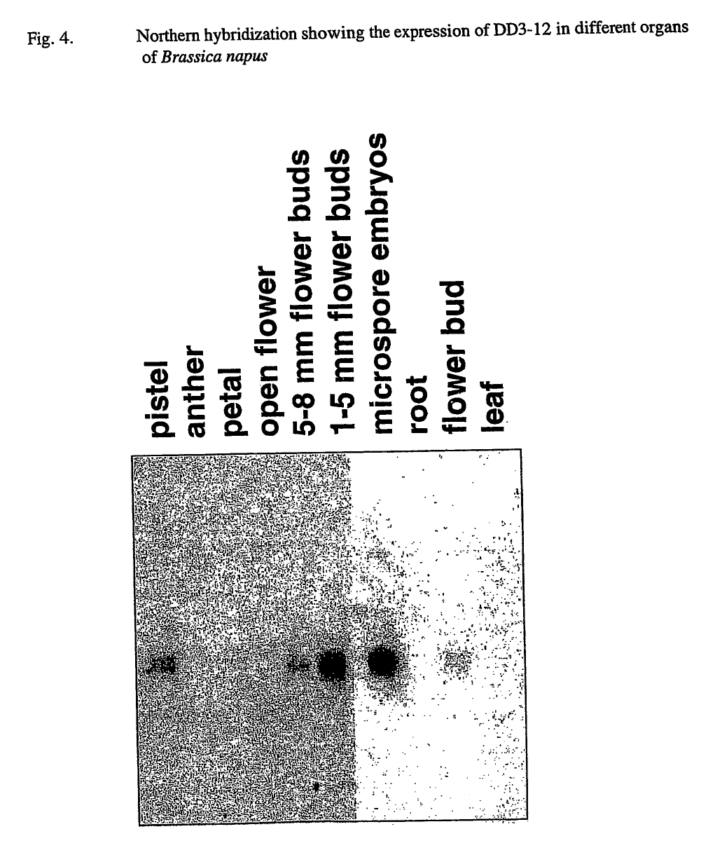

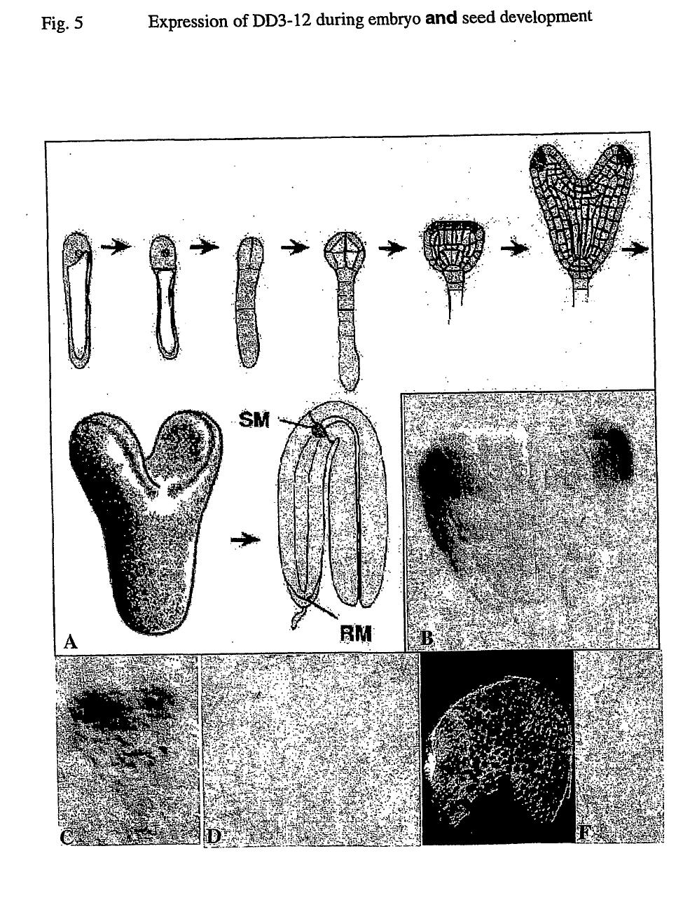

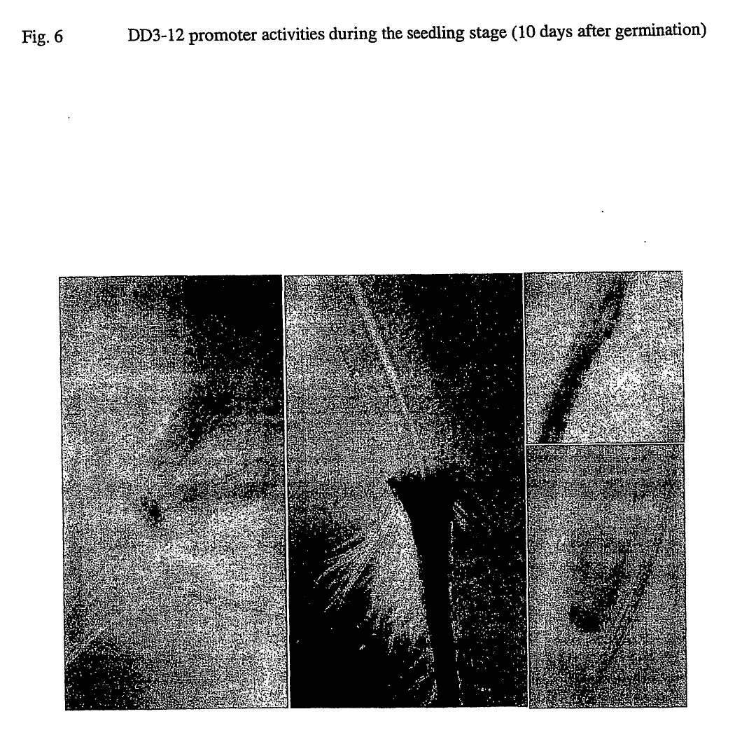

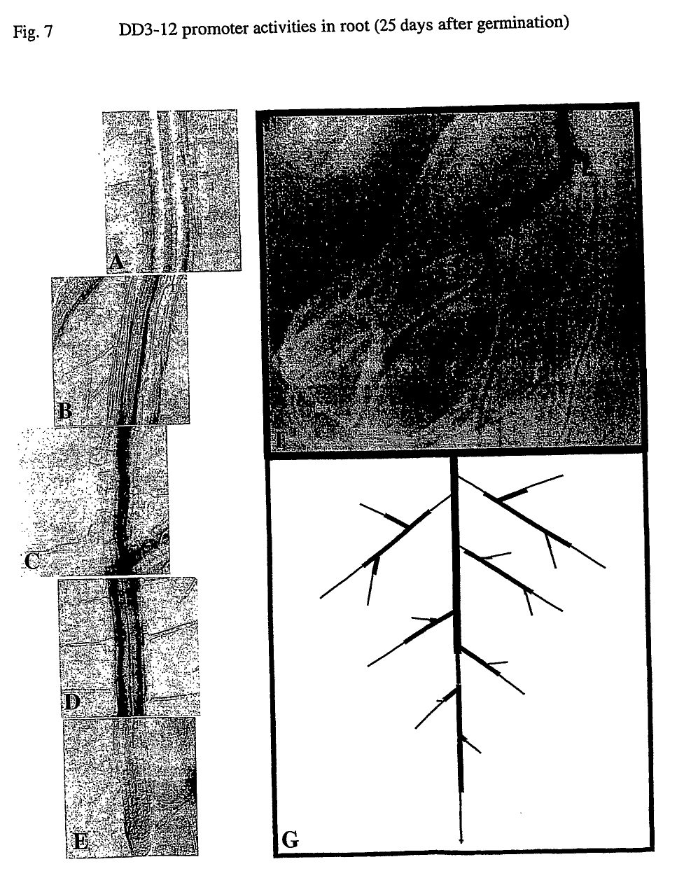

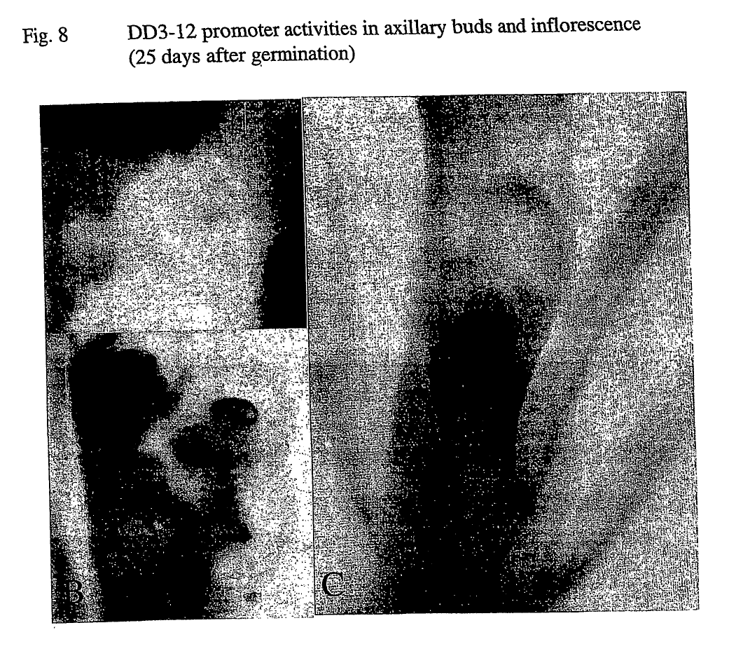

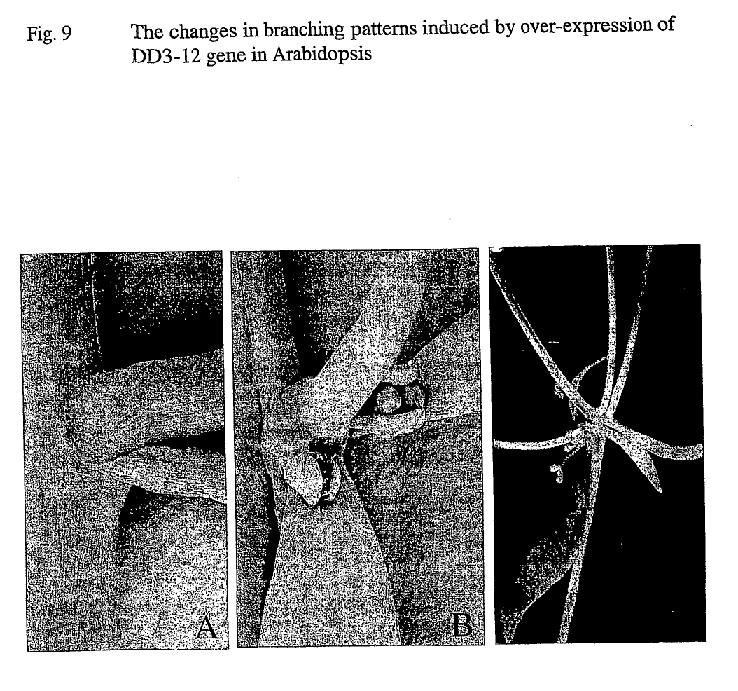

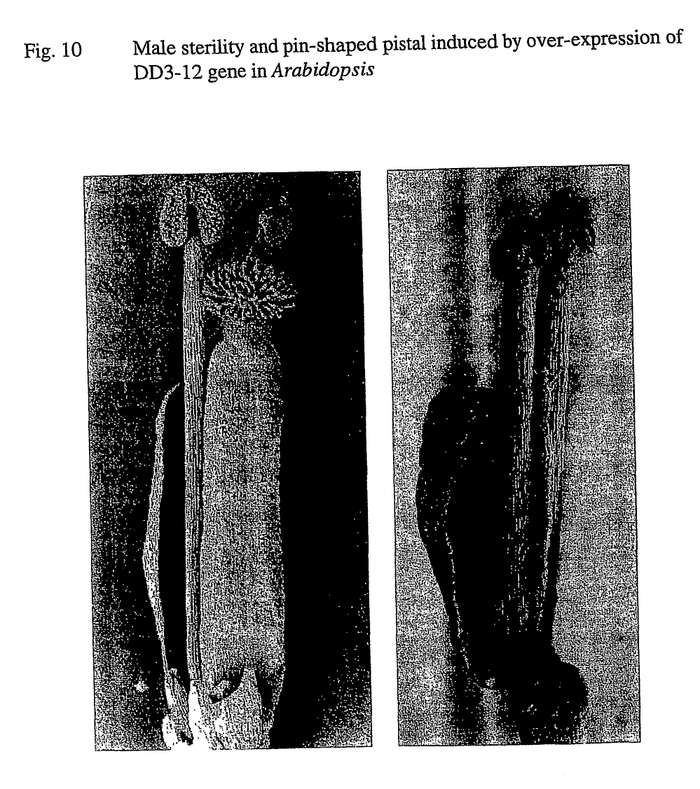

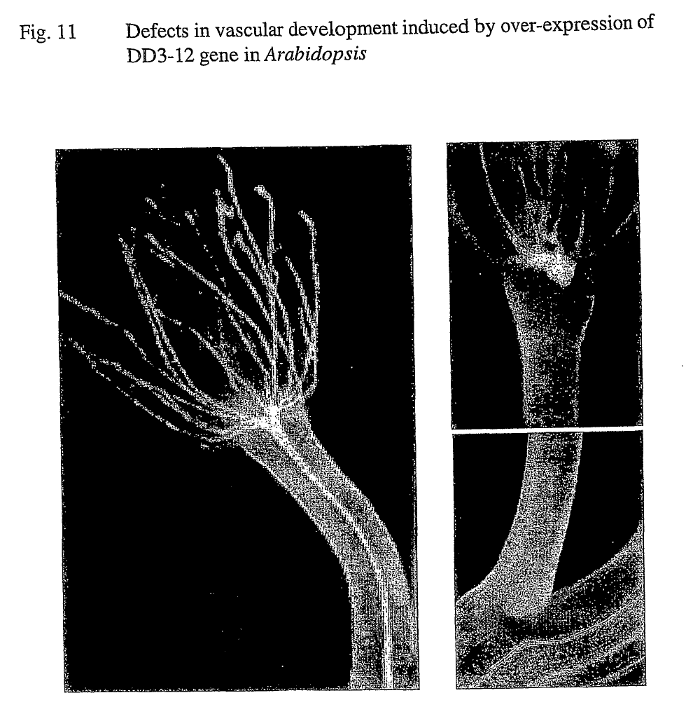

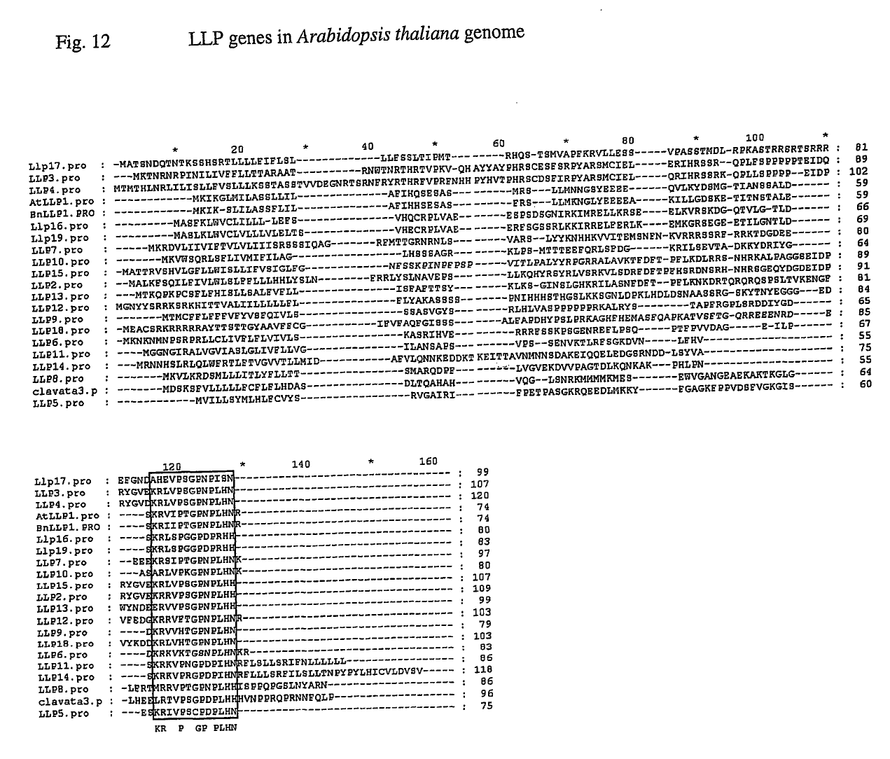

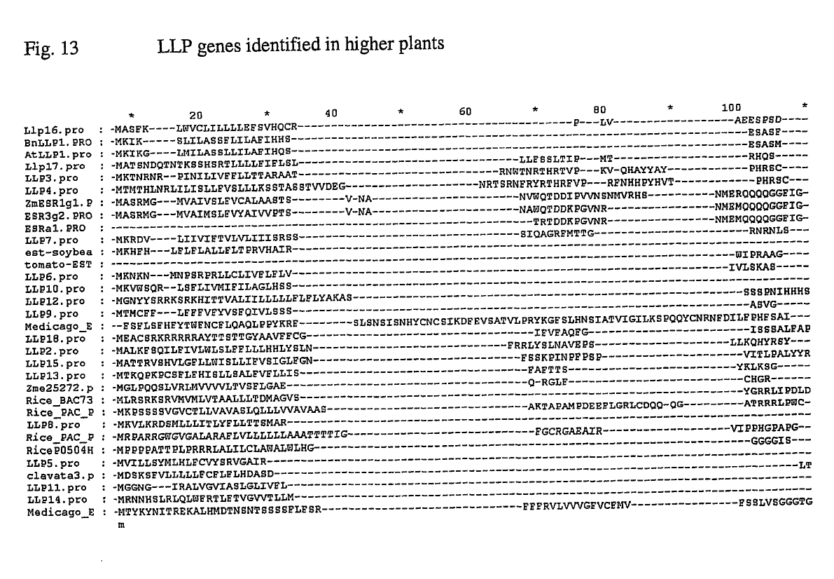

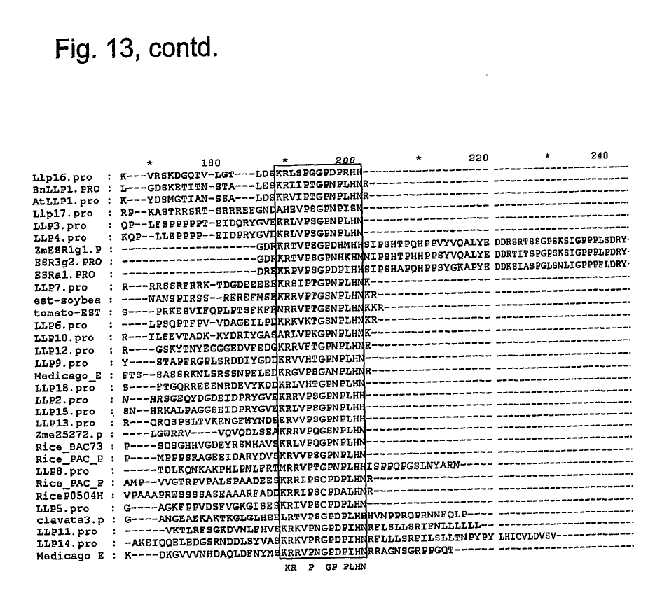

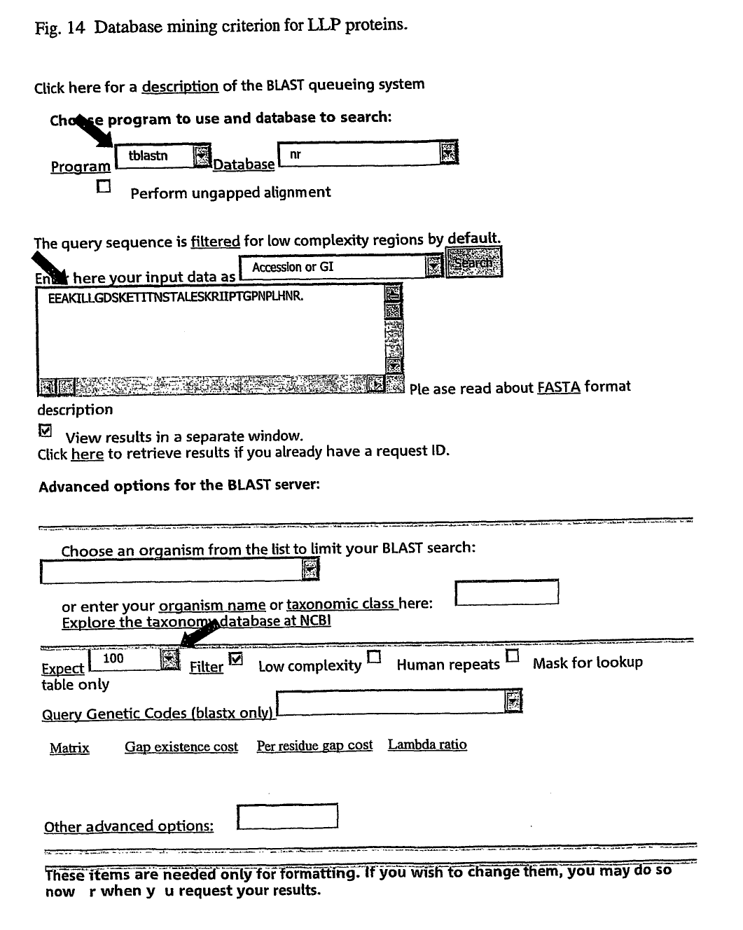

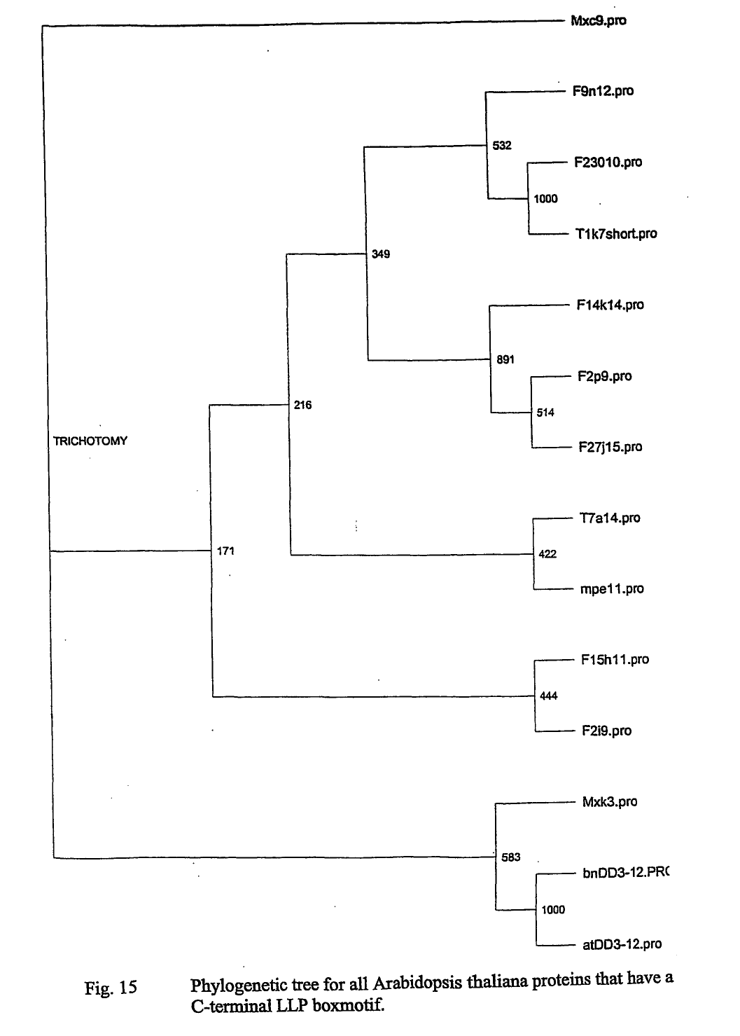

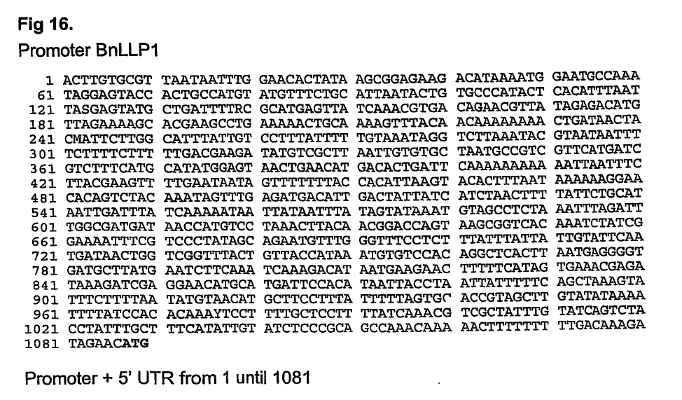

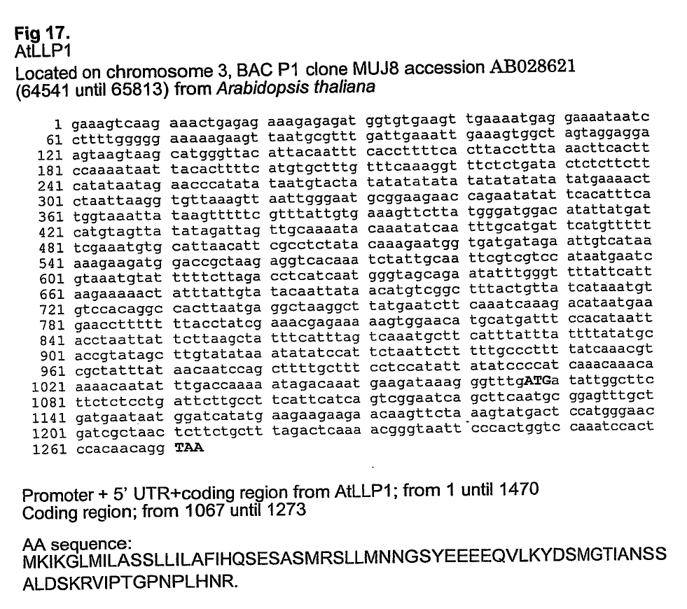

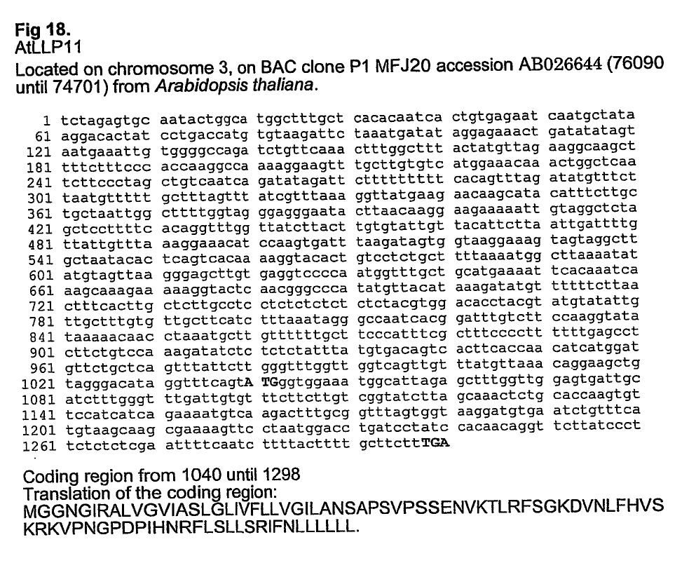

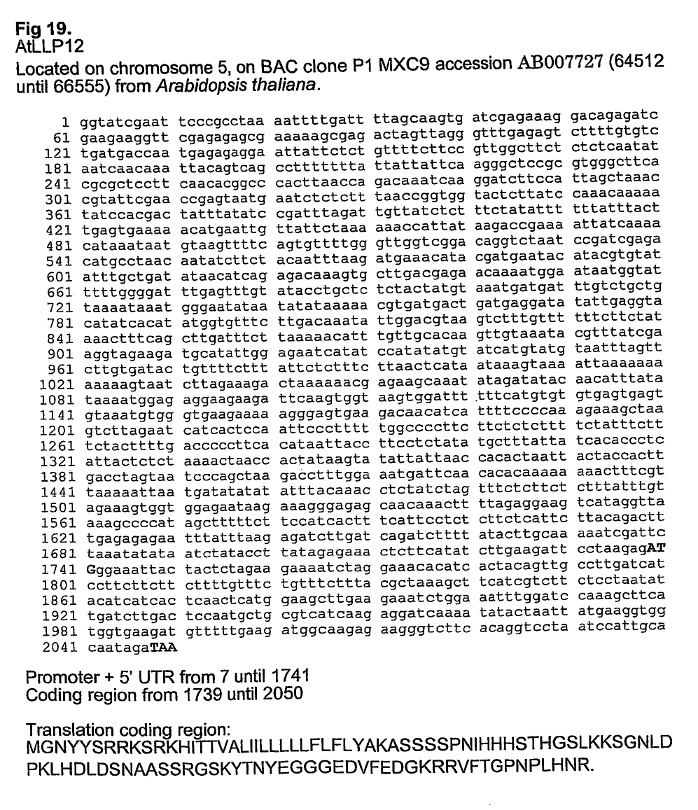

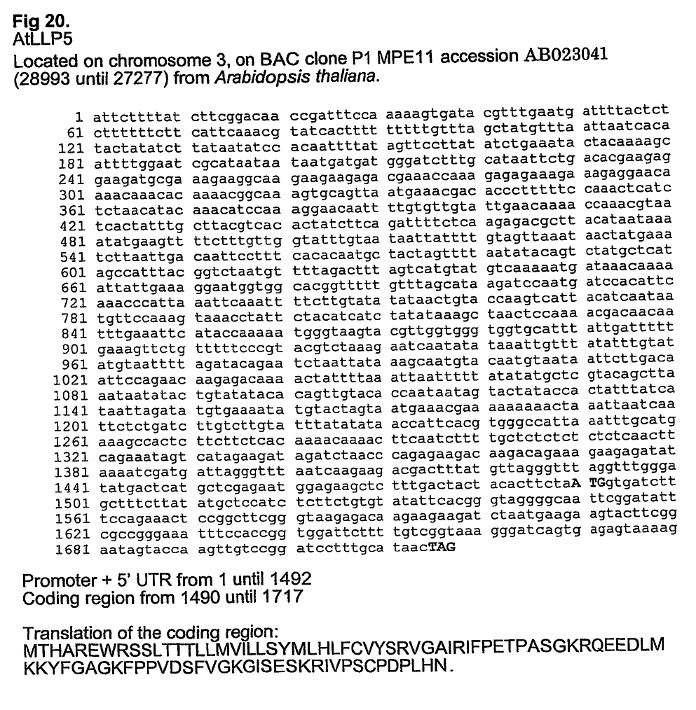

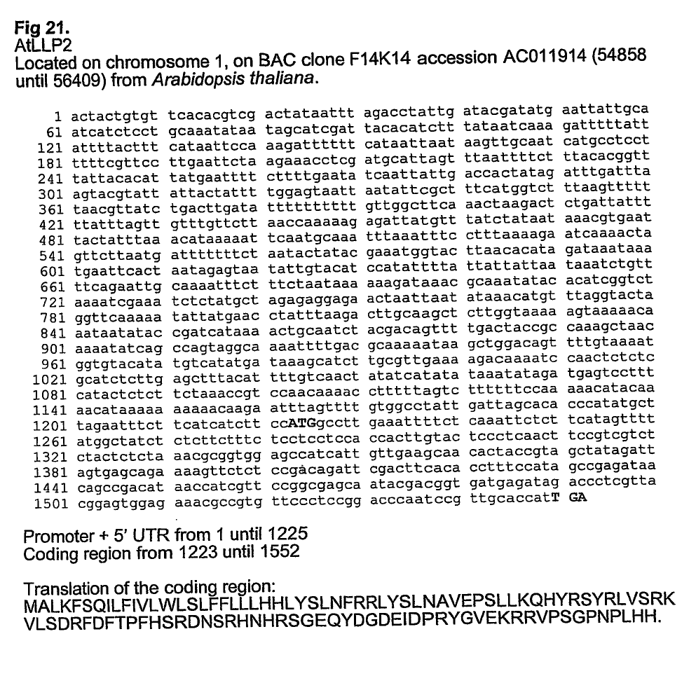

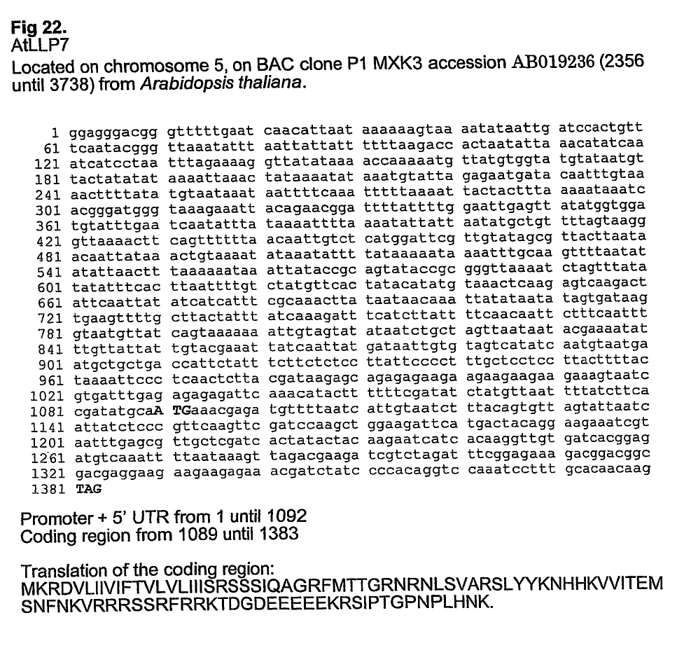

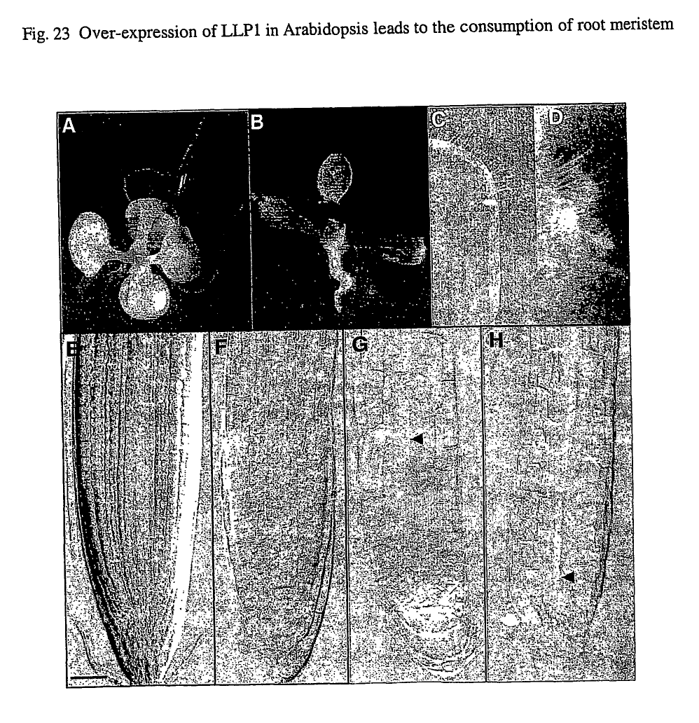

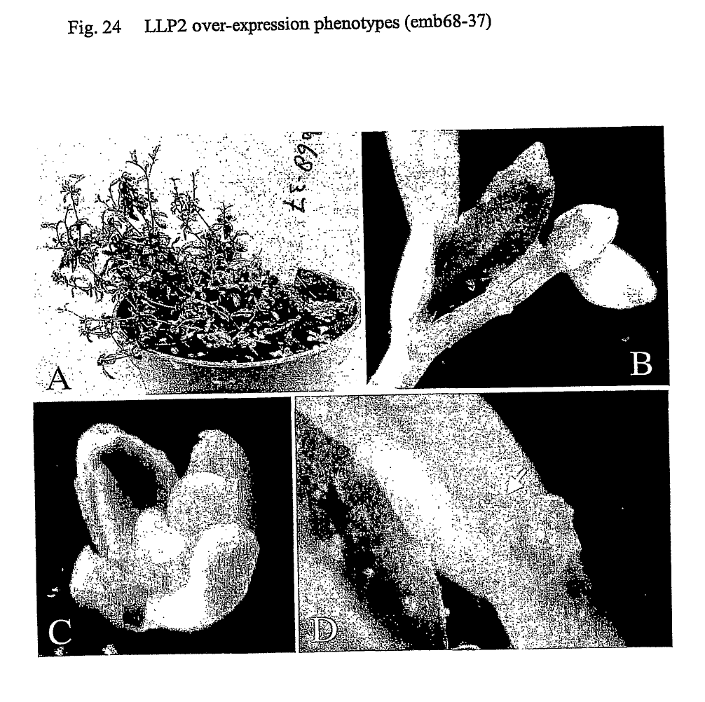

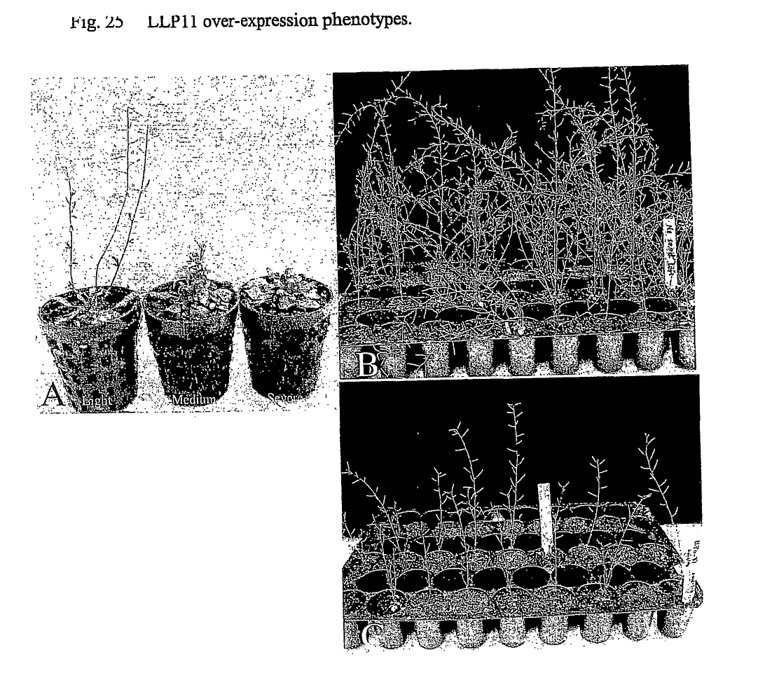

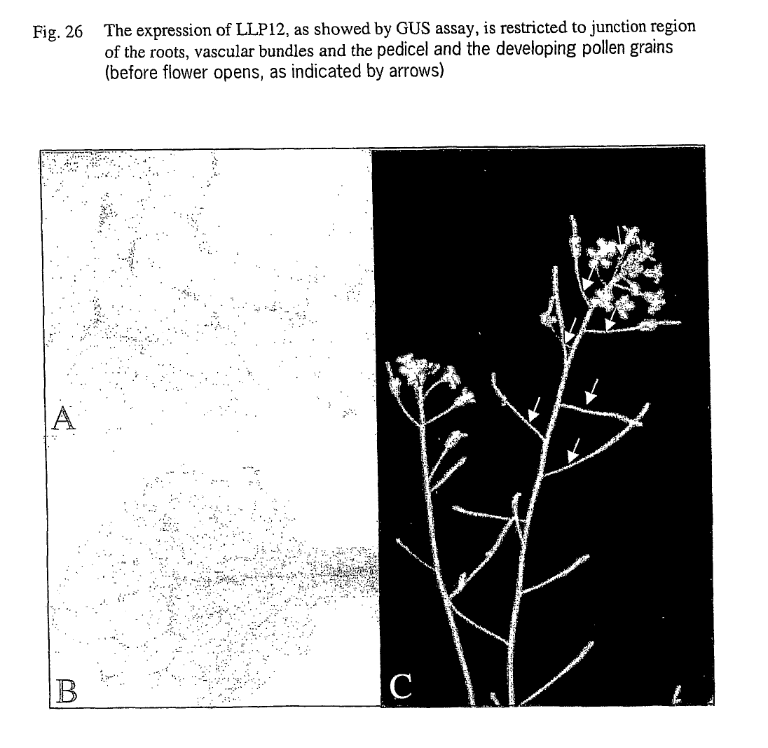

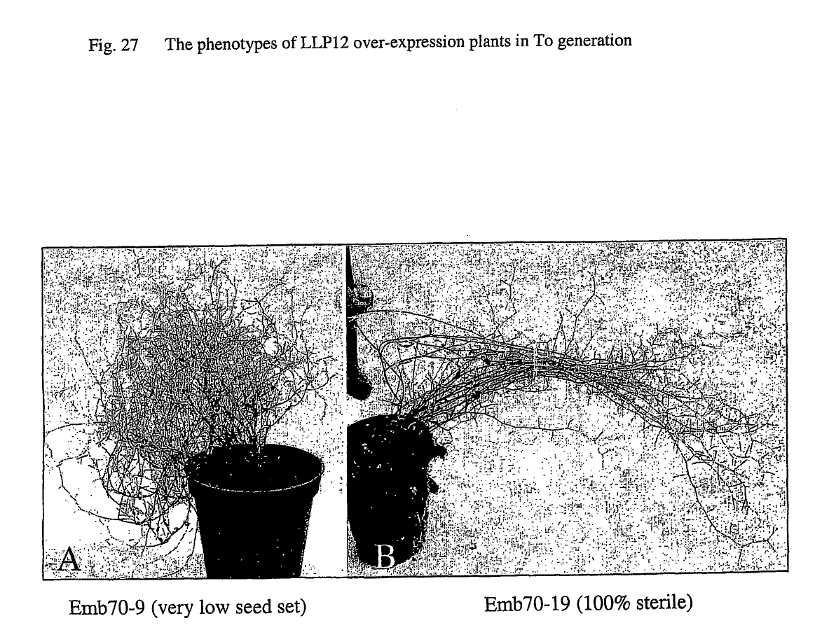

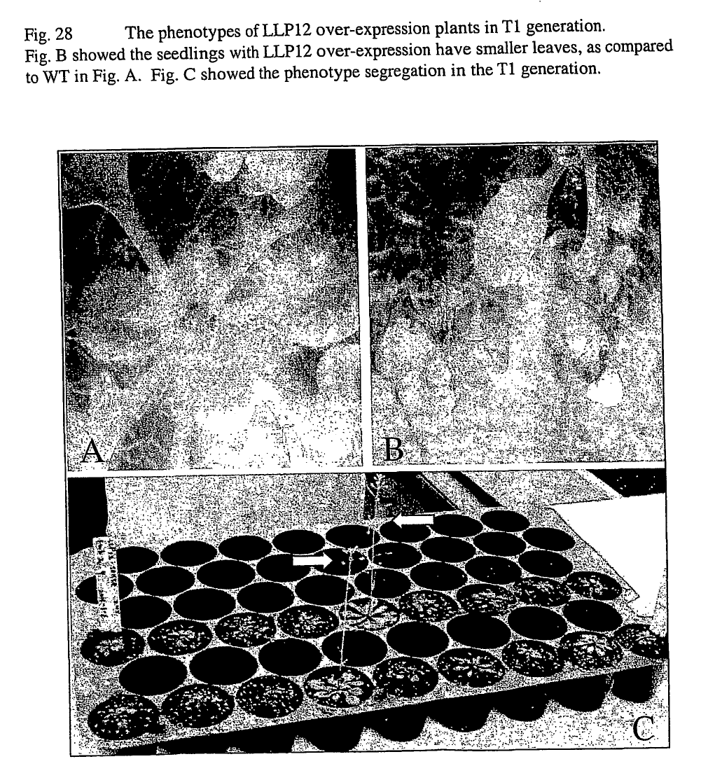

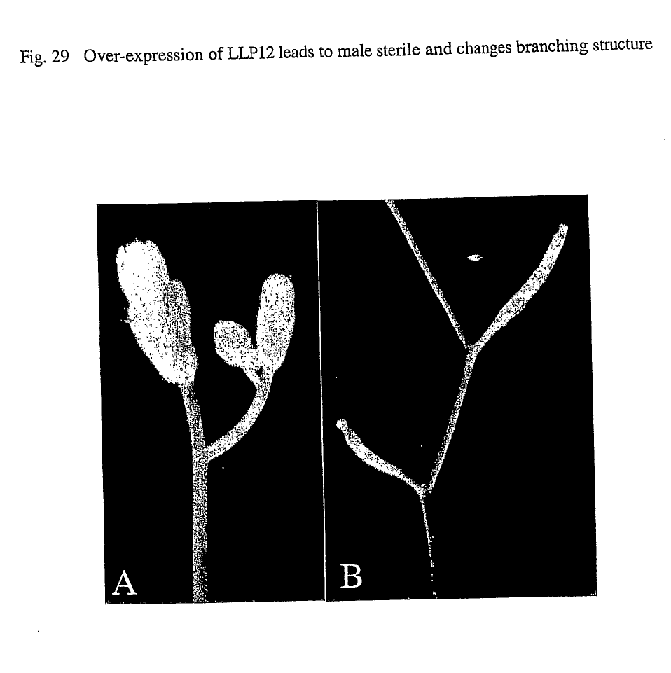

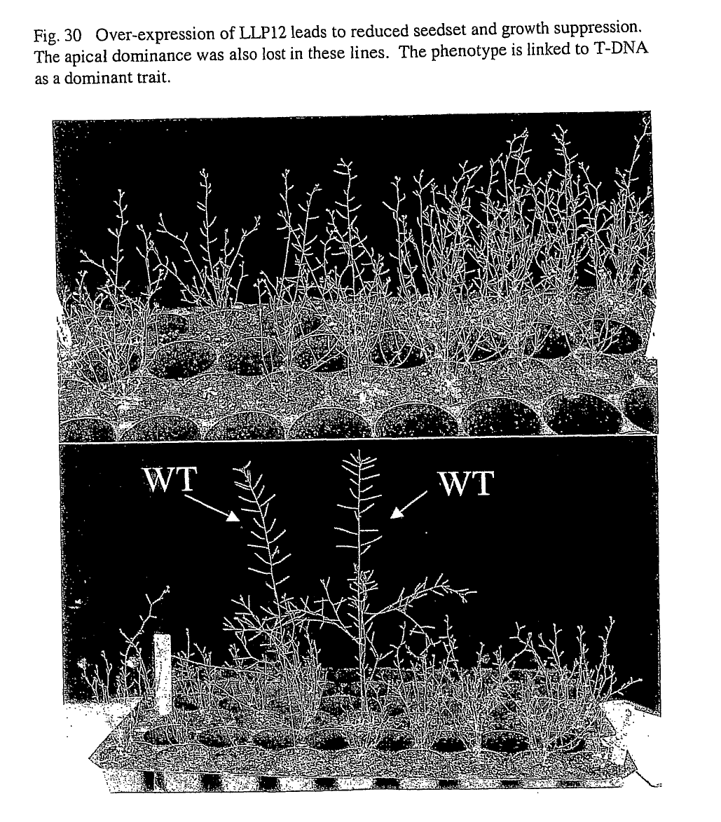

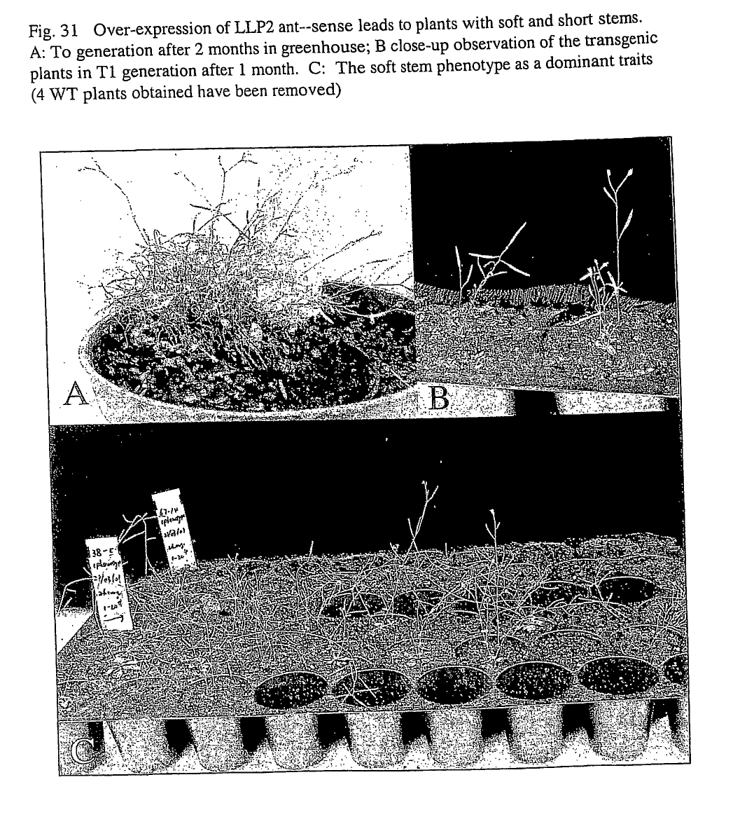

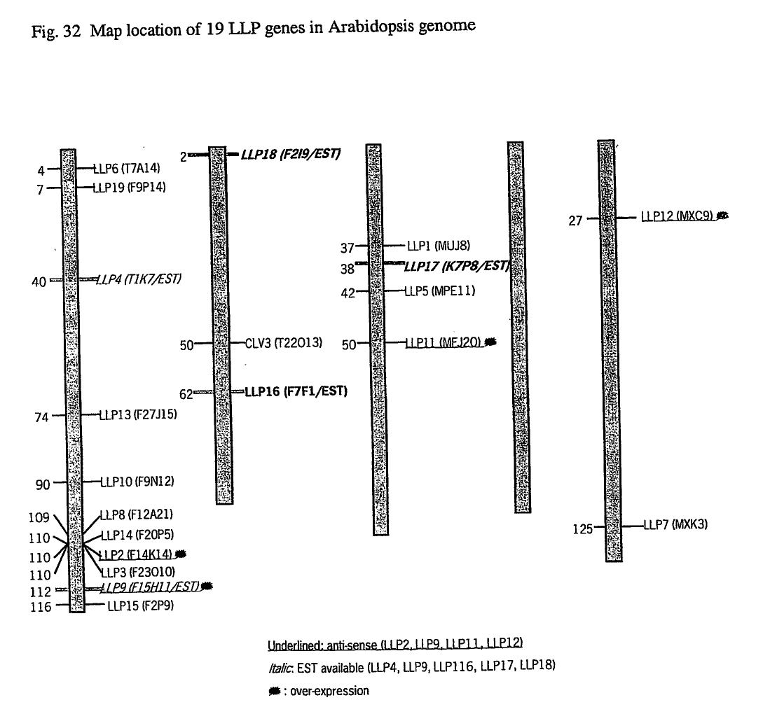

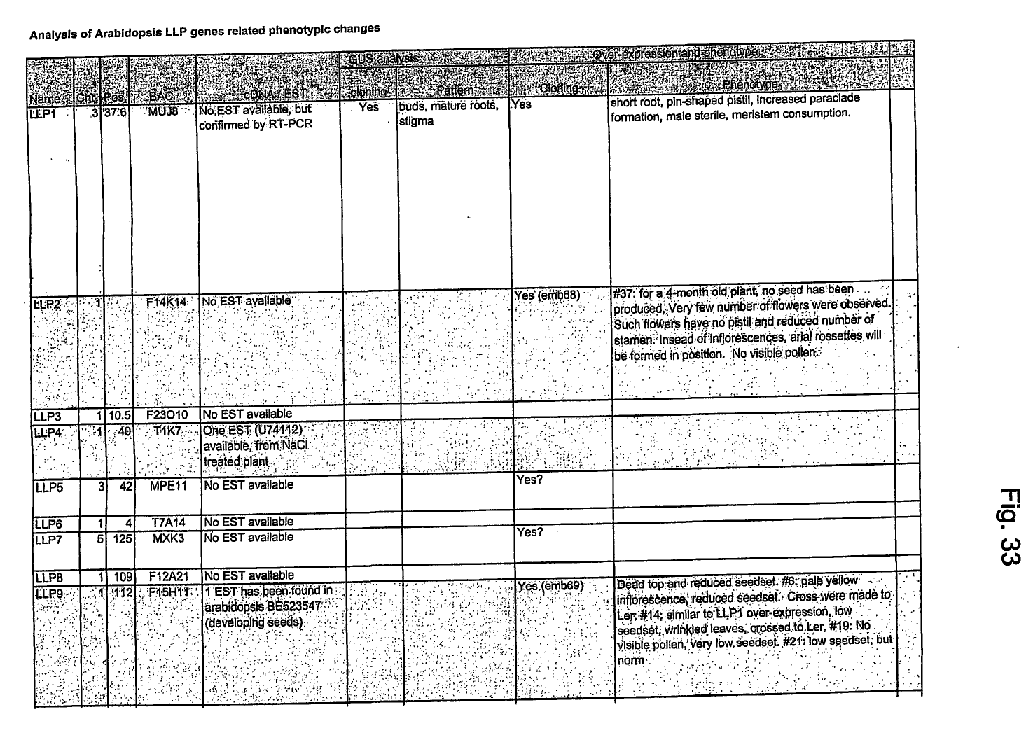

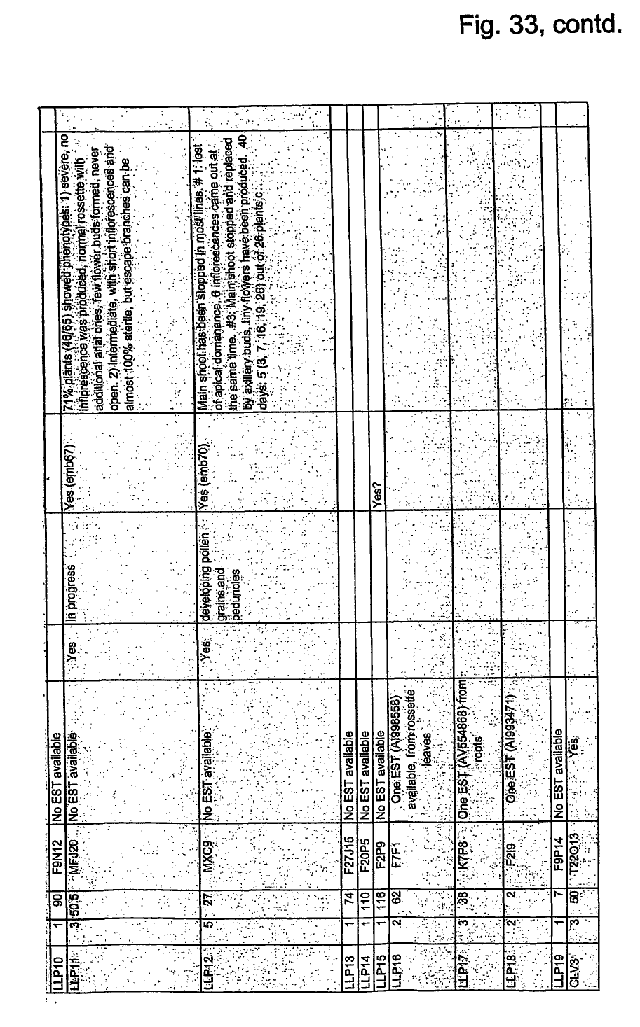

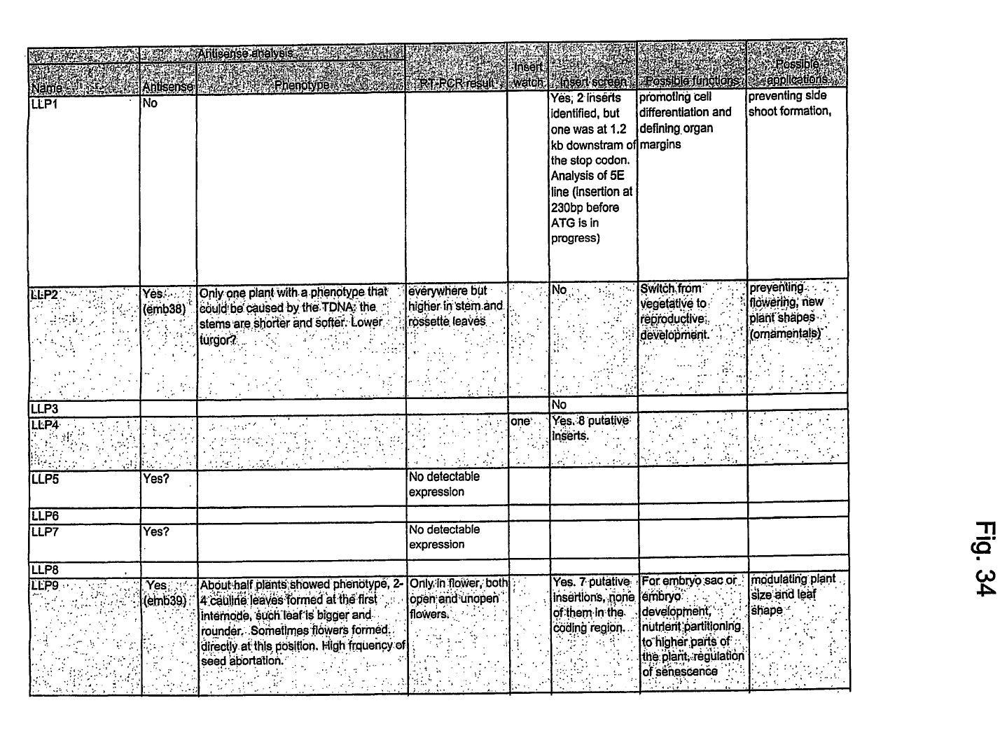

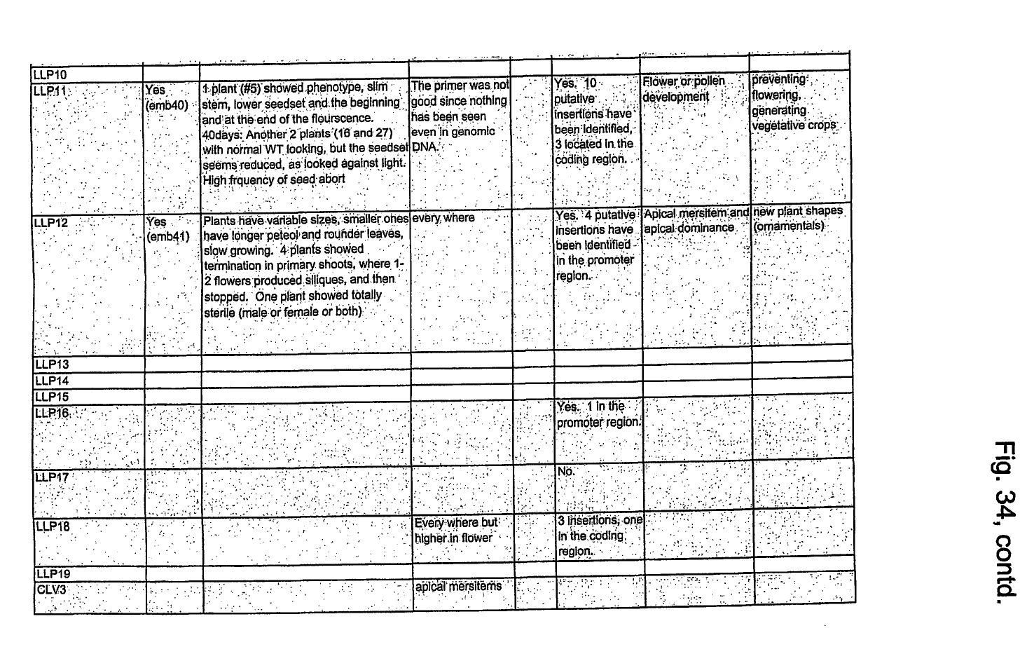

freshly isolated microspores (t=0);

microspores cultured for 8 hr at 18° C. (8 h 18° C.);

microspores cultured for 8 hr at 32° C. (8 h 32° C.);

same as lane 2, but a different RNA isolation;

same as lane 3, but a different RNA isolation;

microspores that were heat-shock treated at 42° C. for 45 minutes (no embryos will be produced from such treatment, 45′ 42° C.);

microspore-derived embryos isolated from 10 days-old culture (10d embryos);

microspore-derived embryos isolated from 16 days-old culture (16d embryos);

leaves (leaf).

Note that, among these 9 RNA samples, the LLP1 signal (indicated by an arrow) was only seen in the lanes where the RNAs were isolated from microspore-derived embryos after 10 and 16 days culture.

FIG. 3 The cDNA and protein sequence of LLP1. The top strand shows the cDNA isolated from a cDNA library of Brassica napus “Topas”, and the bottom strand shows the fragment isolated originally by DD-PCR. The coding region together with the amino acid sequence was underlined, the signal peptide is double underlined, and the LLP boxmotif boxed.

FIG. 4 Northern blot hybridization showing the expression of LLP1 gene in different organs and tissues of Brassica napus “Topas”. Total RNAs were isolated from tissues marked above the gel and hybridised with labelled LLP1 fragment from DD-PCR.

FIG. 5 Expression of LLP1 gene during embryo and seed development in Arabidopsis thaliana.

A. Diagrammatic drawing shows the expression pattern of the LLP1 gene, as revealed by LLP1 promoter::GUS fusion.

B-F. GUS staining of a late globular stage (B) and a heart-shape stage (C), cotyledonary stage (D) embryos and mature seed (E) and seed coat after germination (F). These results were obtained by GUS staining of transgenic plants carrying LLP1 promoter::GUS fusion construct.

The LLP1 gene is expressed firstly in a late globular embryo (as marked in red) and restricted to the top of the cotyledons (as showed in C) and later to the edge of the cotyledon at the torpedo stage. In cotyledonary embryos the expression is restricted to the base of the cotyledon, not in the apical meristem. The expression was switched off in the embryo thereafter. In mature and germinating seeds, the expression is restricted to the remaining endosperm (also called aleurone layer, E and F).

FIG. 6 LLP1 promoter activity in the seedling stage (10 days after germination).

A. Shoot apex;

B. Hypocotyl and root;

C. Main root and lateral root;

The LLP1 gene is not expressed in young seedling within 5 days of germination. In 10-day old seedlings, the LLP1 gene starts to express in the axillary buds (A) and roots with well-established root hairs (B). Such expression was excluded from the epidermal layer and the roothair. Note that no expression was seen in the hypocotyl (B) and the newly formed side roots (C).

FIG. 7 The LLP1 promoter activities in roots. The staining was carried out in seedlings 25 days after seed germination.

A-E. A series pictures taken from one root at different positions. The expression of the LLP1 gene is absence in the root-tip (E), highest in the root hair region (D) and gradually restricted to the vascular bundles (C and D) and disappeared in mature roots (A).

F. A diagrammatic representation of the LLP1 expression in the root system, as indicated in red.

G. Transverse section through the upper part of the root hair region indicating that the expression is mainly in the vascular system.

FIG. 8 LLP1 promoter activities in the axillary buds and the inflorescence (25 days after seed germination).

A. Longitudinal section through a young axillary bud revealing the expression of the LLP1 gene is only in the periphery of the apical meristem.

B. A developing axillary bud showing the promoter activity in the leaf primordia but not in the central meristem.

C. LLP1 gene is not expressed in mature leaves and stems.

D. Young flower buds showing the LLP1 expression in the region between sepal and carpel primordia in young flower buds and then in the stigmatic cells. These cells form a two-lip structure at the beginning and a ring at the later stage.

FIG. 9 Changes of branching patterns in Arabidopsis thaliana “C24” induced by the over-expression of the LLP1 gene under the control of 35S promoter.

A. Electron microscopy photography showing a wild type stem with one shoot normally formed from each axillary bud.

B. Electron microscopy photography showing 3 inflorescences were formed from one axillary bud.

C. At the later stage of plant development, more than 6 shoots could be seen from one axillary bud.

FIG. 10 Male sterility and pin-shaped pistil induced by the over-expression of LLP1 gene in Arabidopsis thaliana “C24”.

A. Wild type flower observed by electron microscopy.

B. Flower from a 35S:LLP1 transgenic plant showing the anther without viable pollen grains and pin-shaped pistil.

No ovule was formed within such a pin-shaped pistil

Some flower organs have been removed when the electron microscopy materials were prepared.

FIG. 11 Defects in vascular development induced by over-expression of LLP1 gene in Arabidopsis thaliana “C24”.

A. A wild type flower showing normal xylem formations

B, C. Flower from LLP1 over-expression plants showing the failure of xylem connection between flower and main stem.

FIG. 12. LLP genes in Arabidopsis thaliana genome. Peptide alignment of the LLP genes identified from Arabidopsis genome. In total 19 LLP genes (1-19) have been found. All peptides encoded by these LLP genes have an N-terminal signal peptide and a C-terminal conserved LLP box. CLV3 and three other LLP proteins have a longer C-terminal span of sequences.

FIG. 13 LLP genes identified in higher plants. Alignment of LLP proteins identified from Arabidopsis and other higher plants. Species with LLP genes include Arabidopsis, tomato, maize, soybean, medicago, and rice. The conserved LLP box is. highlighted in color. Maize ESR proteins have longer C-terminal span after the LLP box.

FIG. 14. Database mining criterion for LLP proteins

FIG. 15. Phylogenetic tree for all Arabidopsis thaliana proteins that have a C-terminal LLP boxmotif.

FIG 16. The promoter sequence of BnLLP1

FIG. 17. AtLLP1: Located on chromosome 3, BAC P1 clone MUJ8 accession B028621 (64541 until 65813) from Arabidopsis thaliana

FIG. 18. AtLLP11: Located on chromosome 3, on BAG clone P1 MFJ20 accession AB026644 (76090 until 74701) from Arabidopsis thaliana.

FIG. 19. AtLLP12: Located on chromosome 5, on BAG clone P1 MXC9 accession B007727 (64512 until 66555) from Arabidopsis thaliana.

FIG. 20. AtLLP5: Located on chromosome 3, on BAG clone P1 MPE11 accession AB023041 (28993 until 27277) from Arabidopsis thaliana.

FIG. 21. AtLLP2: Located on chromosome 1, on BAC clone F14K14 accession AC011914 (54858 until 56409) from Arabidopsis thaliana.

FIG. 22. AtLLP7: Located on chromosome 5, on BAG clone P1 MXK3 accession AB019236 (2356 until 3738) from Arabidopsis thaliana.

FIG. 23 Over-expression of LLP1 in Arabidopsis leads to the consumption of root meristem

A) A wildtype seedling showing the well-developed leaves and roots.

B) A LLP1 over-expression seedling (same age as in A) shows the reduced growth in root. Note the root hairs formed in the short root.

C) A close observation of root from a WT plant showing the normal root morphology.

D) Roots from a LLP1 over-expression plant showing the short and then root with the root hairs formed toward the tip (same magnification as in D).

E) WT root cleared with Hoyer and observed with a DIC microscope to showed the WT root morphology.

F) A root from a 7-day old plant showing the reduced length of the root meristem and the elongation zone.

G) A root from a 10-day old seeding showed the further reduction of root meristem and the elongation zone. The vascular bundle was indicated by an arrowhead.

H) A root from a 14-day old seedling showed the disappearance of root meristem and the elongation zone. The vascular bundle (indicated by an arrowhead) was formed all the way to the central cell region.

FIG. 24 Over-expression of LLP2 prevent reproductive development

A) The phenotype of the LLP2 overexpression plant. Note that no seeds have been produced from the 3-month old Arabidopsis.

B) Close-up observation showing few flowers could be formed occasionally, but no seeds can be produced.

C) The flower formed in the LLP2 over-expression plant produces 2 stamen and no pistil. No pollen was released from the anther. So such plant is both male and female sterile.

D) Inflorescence meristem (indicated by an arrow) was terminated after producing 1-2 abnormal flowers (removed to expose the meristem).

FIG. 25 Over-expression of LLP11 leads to reduction of seed setting

A) Three independent transformants (To generation) showing different degree (light, medium and severe) of phenotypes. Plants with “medium” or “severe” phenotypes produce little or no seeds for further analysis, although the vegetative growth was normal.

B) and C) Progeny analysis of the “light” phenotype plants in the T1 generation. Two types of phenotype were observed: sterility (A) and retarded growth (B). B) Genetic analysis showed that the sterile phenotype is a dominant trait in the T1 generation. A few WT plants obtained from segregation were removed. C) A family of T1 plants shows the phenotype of retarded vegetative and reproductive growth. Only few siliques were produced from each plant. The rosette leaves were also smaller. A few WT plants obtained from segregation were removed.

FIG. 26 The expression pattern of LLP12 gene in Arabidopsis. The result was obtained by analysis of LLP12 promoter::GUS transgenic plants.

A) LLP12 gene was expressed in the junction region of the roots. The expression was limited to the central vascular bundle.

B) LLP12 was expressed in the vascular tissue of the leaves.

C) Diagrammatic drawing to indicate the LLP1 expression in the inflorescence. The expression was seen only in the pedicel (junction between main stem and the flower) and the anther.

FIG. 27 The phenotype of LLP12 over-expression of pants in the To generation. In both To transgenic plants showed here, the primary shoots were stopped early and multiple side shoots were formed afterward (A and B). The plants have very thin and short inflorescence, with no (B) or a few seeds (A) produced. The reduced seedset seemed to be caused by male sterility since seeds can be produced when cross-pollinated with WT pollen. Flower development was normal,

FIG. 28 The phenotype of LLP12 over-expression plants in the T1 generation.

A) WT plants 20 days after generation.

B) LLP12 over-expression plants 20 days after generation, showing the suppression of growth in both shoots and roots.

C) Segregation of LLP12 over-expression plants in the T1 generation, showing the few WT plants (indicated by arrows) were produced from the single insertion line.

The LLP12 over-expression showed suppression of plant growth and development.

FIG. 29 The over-expression of LLP12 leads to male sterile phenotype and changes in flower positioning.

A) Inflorescence of a LLP12 over-expression plant showing the terminal position of flower and the side position of the inflorescence, which is a reverse of the WT morphology.

B) The changes in flower positioning can also been seen when the siliques were formed. Note that the pedicel was also shorter.

FIG. 30 The over-expression of LLP12 leads to reduced seedset and growth suppression. The apicla dominance also lost in these two transgenic lines (T1 generation). The WT plants were removed from the segregation population in the top figure, but not from the bottom figure (indicated).

FIG. 31 Over-expression of LLP12 anti-sense leads to plants with soft and short stems.

A) A plant at To generation showing short inflorescence with few siliques were produced (a 3-month old plant).

B) Plants in T1 generation showed few siliques were produced from each plant (1 month old).

C) Soft stem was a dominant trait in the segregation population.

FIG. 32 Map position of 19 LLP genes in fully sequenced Arabidopsis genome. Note the large cluster of genes observed at the bottom of chromosome 1 and nothing on chromosome 4. None of these 19 genes have been annotated by the genome-sequencing project.

FIG. 33 Analysis of Arabidopsis LLP genes related phenotypic changes.

1. A method for modulating plant phenotype comprising providing a plant with ligand-like protein (LLP) or a functional fragment thereof, said protein or fragment at least comprising a box comprising an amino acid motif XRXXXXGXXXXHX (LLP box).

2. A method according to claim 1 wherein said box comprises an amino acid sequence K R X (V/I) (P/H) (S/T) G (P/S) (N/D) (P/H) (L/I) H (H/N) or a motif at least 80% homologous therewith.

3. A method according to claim 1 or 2 wherein the C-terminus of said box is located at the most from about 10 amino acids away from the C-terminus of said ligand-like protein or functional fragment thereof.

4. A method according to anyone of claims 1 to 3 wherein said ligand-like protein (LLP) or functional fragment thereof comprises an N-terminal signal peptide.

5. A plant or plant material provided with a proteinaceous substance comprising a box comprising an amino acid motif XRXXXXGXXXXHX.

6. A plant or plant material according to claim 5 wherein said box comprises an amino acid sequence K R X (V/I) (P/H) (S/T) G (P/S) (N/D) (P/H) (L/I) H (H/N) or a motif at least 80% homologous therewith.

7. A plant or plant material according to claim 5 or 6 wherein the C-terminus of said box is located at the most from about 10 amino acids away from the C-terminus of said proteinaceous substance.

8. A plant or plant material according to anyone of claims 5 to 7 wherein said substance comprises an N-terminal signal peptide.

9. A plant or plant material according to anyone of claims 5 to 8 wherein said substance comprises at least about 50 amino acids.

10. A plant or plant material according to anyone of claims 5 to 9 wherein said substance comprises at the most about 250 amino acids.

11. A recombinant nucleic acid provided with a nucleic acid encoding a ligand-like protein or functional fragment thereof at least comprising a box comprising an amino acid motif XRXXXXGXXXXHX, or a nucleic acid hybridising therewith.

12. A recombinant nucleic acid according to claim 11 functionally linked with a promoter.
